(19) United States
(12) Patent Application Publication
(10) Pub. No.: US 2023/0266340 A1
(43) Pub. Date: Aug. 24, 2023

(54) Measurement, Diagnosis, Treatment and Management of Metabolic Syndrome

(71) Applicant: FERNANDO GARCÍA SADA, San Pedro Garza Garcia (MX)

(72) Inventors: Santos Eduardo Flores López, Santa Catarina (MX); Sergio de Jesus Elizondo, San Pedro Garza Garcia (MX); Eduardo Garcia Alanis, San Pedro Garza Garcia (MX); Bernardo Dychter Cholow, Monterrey (MX); Fernando Garcia Sada, San Pedro Garza Garcia (MX); Ivan Pacheco Espino, Monterrey (MX); Alejandro Pulido Watts, Monterrey (MX)

(21) Appl. No.: 17/679,927

(22) Filed: Feb. 24, 2022

(51) Int. Cl. G01N 33/68; G01N 33/543

(52) U.S. Cl. CPC G01N 33/6893; G01N 33/6872; G01N 33/54366; G01N 2333/72; G01N 2800/04; G01N 2800/52

(57) ABSTRACT

The present disclosure refers to a method for the measurement, diagnosis, treatment, and management of metabolic syndrome comprising the administration of a drug for the treatment of metabolic syndrome and a system for monitoring a drug product to a patient in need thereof, to prevent or reduce the risk of metabolic syndrome and related disorders, by using electronic devices.

### BACKGROUND

### Technical Field

The present disclosure refers to a useful method for the measurement, diagnosis, treatment and management of metabolic syndrome comprising the administration of a drug product to prevent or reduce the risk of metabolic syndrome and related disorders, by means of telemedicine and dispensing of drug products and supplies from the use of a user interface that allows intercommunication with measuring devices associated with a set of essential elements to meet definite purpose between the physician and/or the patient.

### Description of Related Art

Metabolic syndrome is a group of risk factors that occurs when a person has at least three of the following parameters: waist circumference of more than 40 inches in men and 35 inches in women; triglycerides above 150 milligrams per deciliter of blood (mg/dL); high-density lipoprotein (HDL) cholesterol at less than 40 mg/dL in men or less than 50 mg/dL in women; high fasting glucose of 100 mg/dL or more; systolic blood pressure of 130 mmHg or more and/or diastolic blood pressure of 85 mmHg or more, resulting in an increased risk of cardiovascular disorders, diabetes, blood clots, fatty liver disease, among other health problems.

The presence of metabolic syndrome can arise from lack of physical activity, overweight, obesity or genetic conditions and the lack of treatment or control results in the physical deterioration of the patient, as well as a negative impact on their physical performance in daily activities.

Especially, overweight and obesity are defined as an abnormal or excessive accumulation of fat that can be detrimental to health. Currently, there are more than 1.9 billion people over 18 years of age who are overweight, of which 650 million have some degree of obesity, while there are more than 380 million people under 18 years of age who are overweight or obese, which is why the World Health Organization has classified it as a global pandemic.

According to the CDC (Centers for Disease Control and Prevention), the Body Mass Index (BMI) is the weight of a person in kilograms divided by the square of height in meters, being a high BMI a possible indicator of high body fat.

In this regard, the CDC presents the following BMI ranges to identify the presence of overweight and obesity:
BMI less than 18.5, falls within the underweight range;
BMI of 18.5 to <25, falls within the healthy weight range;
BMI of 25.0 to <30, falls within the overweight range;
BMI of 30.0 or higher falls into the obese range.

Additionally, obesity is subdivided into 3 classes depending on BMI:
Class 1: BMI 30 to <35;
Class 2: BMI of 35 to <4;
Class 3: BMI of 40 or higher. “Severe” obesity.

Overweight people, compared to those with a healthy weight, have an increased risk of many serious diseases and health conditions, including the following: high blood pressure (hypertension), high LDL cholesterol, low HDL cholesterol or high triglyceride levels (dyslipidemia), type 2 diabetes, coronary heart disease, stroke, gallbladder disease, osteoarthritis (a breakdown of cartilage and bone within a joint), sleep apnea and respiratory problems, several types of cancer, poor quality of life, mental illness such as clinical depression, anxiety and other mental disorders, bodily pain and difficulty in physical functioning, and others.

As can be seen, obesity and overweight are the major risk factor for developing metabolic syndrome; however, it also corresponds to a factor that is easily detectable, and it can be prevented or controlled in any of its stages.

Access to health care professionals specializing in metabolic syndrome and related disorders is complicated because it is usually done on recommendation; or no contact is made at all with such health care professionals, opting for self-medication or the use of remedies or exercise equipment that promise weight reduction or improve the patient health. Added to this is the lack of time/willingness of patients to go to the physician and the availability of physician time to be able to adequately attend to the patient; as well as the lack of time/willingness of patients for their treatment and the supplier availability in inventory to provide it; resulting in the patient suspending or leave the treatment.

Multiple tools are available for weight control or weight loss; however, patients generally resort to diets, sports routines, or treatments without proper planning or without the help of expert health care professionals, resulting in patient torture, in excessive weight gain once a diet is over or in damage to the patient health that can lead to death.

In the case of the treatment of overweight and obesity using dietary supplements or drug products, it is essential the participation of a health care professional to monitor and involve throughout the treatment to avoid damage to the patient health.

Difficulty in maintaining preventive treatment or control of overweight and obesity, as well as other factors of metabolic syndrome, compromises patient health, therefore, it is necessary a monitoring system during the administration of the drug product, with continuous access to the health care professional and with a system of assortment of the drug product or drug products to meet several objectives: to ensure the supply of drug products, supplements, supplies or devices for quality and safe measurement; to provide continuity and motivation to maintain the treatment; and to monitor in real time with the help of technology the parameters that allow the health care professional to know the progress of the treatment to evaluate whether it is necessary to modify the therapeutic scheme or to maintain it.

### SUMMARY

Accordingly, provided is a useful method for the measurement, diagnosis, treatment, and management of metabolic syndrome comprising the administration of a drug product to a patient in need thereof, to prevent or reduce the risk of metabolic syndrome and related disorders, by using electronic devices, thus allowing the assignment of an adequate treatment, as well as the supply of drug products, supplies and devices required for real-time monitoring by a health care professional specialist.

As particular objectives, the present disclosure provides different tools to facilitate adherence to the treatment, followed by continuous recommendations regarding routines, diet, periodicity of medical appointments, exercise, among other activities that allow strengthening the treatment and its monitoring by the health care professional.

Another particular objective of the present disclosure is to maintain: a constant connectivity between the patient and the health care professional, avoiding breaking with communication of data in both respects; a constant connectivity between the patient and the drug product provider, supplies or required devices, avoiding the treatment abandoning due to lack thereof, even notifying and reminding the patient before any of them is finished; and, a connectivity between the patient and the measuring devices, avoiding the need for transcription of data or errors that may occur when doing so.

### BRIEF DESCRIPTION OF THE DRAWINGS

FIG. 1 is a graphical representation of a method for the measurement, diagnosis, treatment, and management of metabolic syndrome;

FIG. 2 illustrates a non-limiting embodiment or aspect of a method for the measurement, diagnosis, treatment, and management of metabolic syndrome comprising the administration of a drug product to a patient in need thereof, to prevent or reduce the risk of metabolic syndrome and related disorders according to the present disclosure;

FIG. 3 illustrates a non-limiting embodiment or aspect of a method for the measurement, diagnosis, treatment, and management of metabolic syndrome comprising the administration of a drug product to a patient in need thereof, to prevent or reduce the risk of metabolic syndrome and related disorders according to the present disclosure;

FIG. 4 illustrates a non-limiting embodiment or aspect of a system to monitor at least one clinical parameter and/or anthropometric indicator to a patient in need thereof, to prevent or reduce the risk of metabolic syndrome and related disorders according to the present disclosure;

FIG. 5 illustrates a non-limiting embodiment or aspect of a method for the measurement, diagnosis, treatment, and management of metabolic syndrome comprising the administration of a drug product to a patient in need thereof, to prevent or reduce the risk of metabolic syndrome and related disorders according to the present disclosure;

FIG. 6 illustrates a non-limiting embodiment or aspect of a system to monitor at least one clinical parameter and/or anthropometric indicator in a patient in need thereof, to prevent or reduce the risk of metabolic syndrome and related disorders, by using electronic devices associated with devices for measuring clinical parameters and/or anthropometric indicators according to the present disclosure; and

FIG. 7 illustrates the connectivity model of the monitoring system connectivity of at least one clinical parameter and/or anthropometric indicator to a patient in need thereof, to prevent or reduce the risk of metabolic syndrome and related disorders, by a health care professional according to the present disclosure.

### DETAILED DESCRIPTION

The present disclosure refers to a useful management method for the diagnosis, measurement and treatment of metabolic syndrome that comprises the administration of a drug product to a patient in need thereof, to prevent or reduce the risk of metabolic syndrome and related disorders, using electronic devices, the method comprising: (a) collecting and storing patient identity information and/or body metrics information in a database, for incorporation into a medical record; (b) linking the patient with a health care professional specialist in metabolic syndrome and related disorders for the assessment of their health status; (c) indicating to the patient their health status and recording in the medical record; (d) instructing the patient on the indications for the treatment of metabolic syndrome and/or related disorders that he suffers or for the prevention of metabolic syndrome and/or related disorders; (e) providing a patient in need thereof, a pharmaceutical composition with a useful drug product for the prevention, reduction, or control of the metabolic syndrome and/or related disorders; (f) providing a patient in need thereof, supplies or supportive devices for the prevention, reduction, or control of metabolic syndrome and/or related disorders; (g) connecting using an interactive user interface one or more devices for measuring clinical parameters and/or anthropometric indicators; (h) measuring, recording, and storing in a database the clinical parameters and/or anthropometric indicators collected; (i) monitoring the data of the clinical parameters and/or anthropometric indicators of the patient by a health care professional specialist in metabolic syndrome and related disorders, for the assessment of their health status; (j) adjusting or maintaining the indications for the treatment of metabolic syndrome and/or related disorders suffered by the patient or for the prevention of metabolic syndrome and/or related disorders; (k) optionally, assisting to the patient by the health care professional, at set periods of time or at the request of the patient or the health care professional.

The following is a description of illustrative and non-limiting embodiments or aspects of the present disclosure consistent with the description, the “illustrative and non-limiting embodiments or aspects” include the particular aspect, structure or feature which in turn may be integrated by other illustrative and non-limiting embodiments or aspects which are or are not described, therefore the illustrative and non-limiting embodiments or aspects described below are not limiting; therefore, different software or hardware embodiments, interfaces, means of execution, storage or reading, instructions, etc. may be implemented.

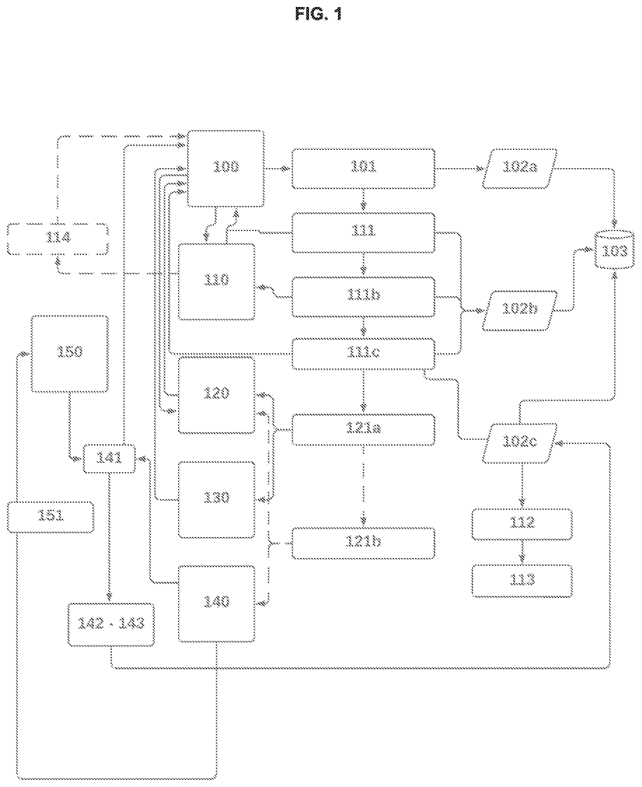

FIG. 1 is a graphical representation of non-limiting embodiments or aspects of a useful method for the measurement, diagnosis, treatment, and management of metabolic syndrome comprising administering a drug product to a patient (100) requiring it, to prevent or reduce the risk of metabolic syndrome and related disorders, using of electronic devices, the method comprising: collecting (101) and storing patient identity information (102a) and/or body metrics information (102c) in a database (103), for incorporation into a medical report (102b); linking (111) the patient (100) with a health care professional (110) specialist in metabolic syndrome and related disorders, for the assessment of their health status; indicating the patient his health status (111b) and record in the medical record (102b); instructing the patient (111c) in the indications for the treatment of metabolic syndrome and/or related disorders that he suffers or for the prevention of metabolic syndrome and/or related disorders; through a pharmacy (120), providing (121a) to a patient (100) in need thereof, a pharmaceutical composition with a useful drug product (130) for the prevention, reduction, or control of metabolic syndrome and/or related disorders; through a pharmacy (120), providing (121b) to a patient (100) in need thereof, supplies (140a) or measuring devices (140) for the prevention, reduction, or control of metabolic syndrome and/or related disorders; connecting (151) using an interactive user interface (150) one or more devices for measuring clinical parameters and/or anthropometric indicators (140b); measuring (141), recording (142) and storing (143) in the database (103) the clinical parameters and/or anthropometric indicators collected (102c); monitoring (112) the data of the clinical parameters and/or anthropometric indicators (102c) of the patient (100) by a health care professional (110) specialist in metabolic syndrome and related disorders, for the assessment of their health status; adjusting or maintaining the indications (113) for the treatment of metabolic syndrome and/or related disorders suffered by the patient or for the prevention of metabolic syndrome and/or related disorders; optionally, assisting (114) to the patient (100) by the health care professional (110), at set periods of time or at the request of the patient or the health care professional.

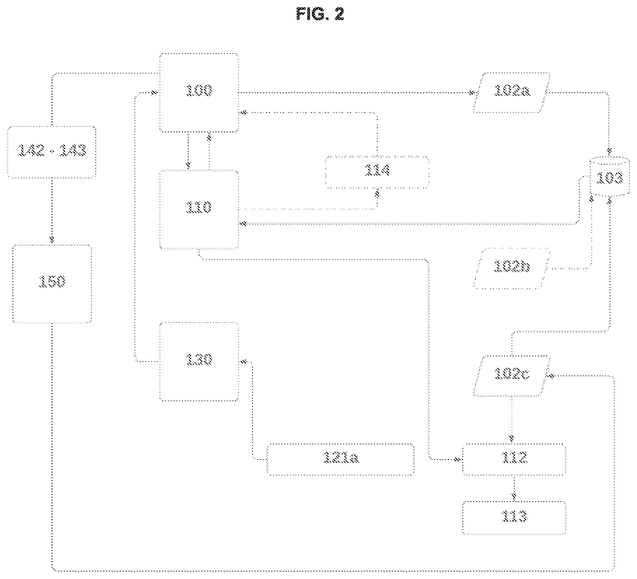

FIG. 2 illustrates non-limiting embodiments or aspects of a useful method for the measurement, diagnosis, treatment, and management of metabolic syndrome comprising the administration of a drug product to a patient in need thereof, to prevent or reduce the risk of metabolic syndrome and related disorders, by using electronic devices, the method comprising: providing (121a) to the patient (100) a pharmaceutical composition with a useful drug product (130) for the prevention, reduction, or control of metabolic syndrome and/or related disorders, from patient identity information (102a) and body metrics information (102c) and/or a medical record (102b) of the patient, previously stored in a database (103); monitoring (112) the data of the clinical parameters and/or anthropometric indicators of the patient by a health care professional specialist (110) in metabolic syndrome and related disorders, for the assessment of their health status through an interactive user interface (150) connected to one or more devices for measuring clinical parameters and/or anthropometric indicators, wherein the interactive user interface (150) records (142) and stores (143) clinical parameters and/or anthropometric indicators.

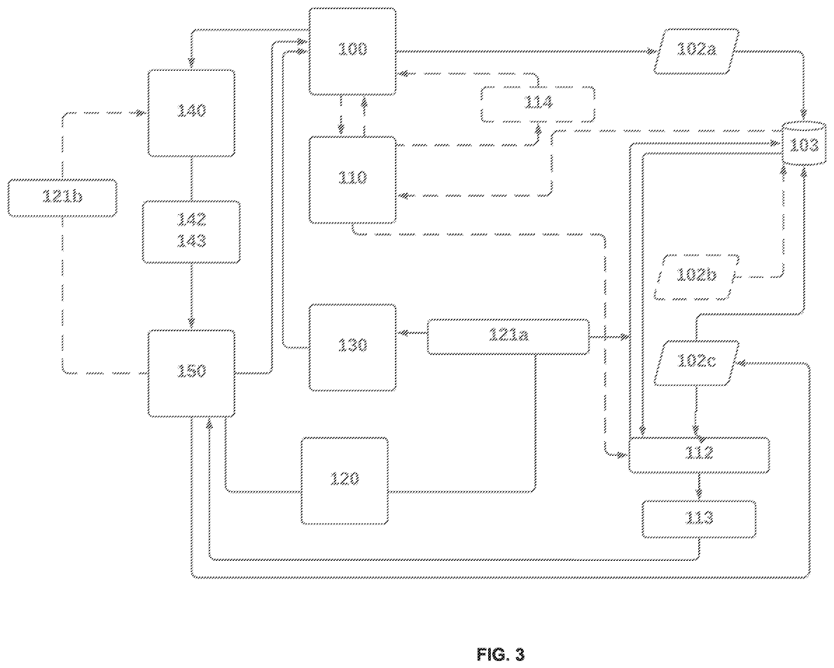

FIG. 3 illustrates non-limiting embodiments or aspects of the useful method for the measurement, diagnosis, treatment, and management of metabolic syndrome comprising the administration of a drug product to a patient in need thereof, to prevent or reduce the risk of metabolic syndrome and related disorders, by using electronic devices.

In non-limiting embodiments or aspects, the method for the measurement, diagnosis, treatment, and management of metabolic syndrome comprising the administration of a drug product in a patient who is undergoing treatment prevention, reduction, or control of metabolic syndrome and/or related disorders, by using of electronic devices, the method comprising: collecting (101) and storing patient identity information (102a) and/or information of body metrics, clinical parameters and/or anthropometric indicators (102c) in a database (103), for the incorporation of a medical record (102b); through a pharmacy (120), providing (121a) to a patient (100) in need thereof a pharmaceutical composition with a useful drug product (130) for the prevention, reduction, or control of metabolic syndrome and/or related disorders, from the identity information of the patient (102a) and the information of the clinical parameters and/or anthropometric indicators of the patient (102c) and/or a medical record (102b) of the patient, previously stored in a database; measuring (141), recording (142) and storing (143) in the database (103) the clinical parameters and/or anthropometric indicators (102c) collected from the patient during treatment; monitoring (112) the data of the clinical parameters and/or anthropometric indicators (102c) of the patient (100) by a health care professional (110) specialist in metabolic syndrome and related disorders, for the assessment of their health status, to adjust or maintain the indications (113) for the treatment of metabolic syndrome and/or related disorders suffered by the patient or for the prevention of metabolic syndrome and/or related disorders.

In non-limiting embodiments or aspects, the useful method for the measurement, diagnosis, treatment, and management of metabolic syndrome comprising the administration a drug product to a patient in need thereof to prevent or reduce the risk of metabolic syndrome and related disorders, by using electronic devices, the method comprising: linking (111) the patient (100) with a health care professional (110) specialist in metabolic syndrome and related disorders, for the assessment of their health status; indicating to the patient his health status (111b) and recording in the medical record (102b); instructing the patient (111c) in the indications for the treatment of metabolic syndrome and/or related disorders that he suffers or for the prevention of metabolic syndrome and/or related disorders; through a pharmacy (120), providing (121a) to a patient (100) in need thereof, a pharmaceutical composition with a useful drug product (130) for the prevention, reduction, or control of metabolic syndrome and/or related disorders; recording (142) and storing (143) in database (103) body metrics, clinical parameters and/or anthropometric indicators collected (102c) from the patient during treatment; recording and storing in a database the clinical parameters and/or anthropometric indicators of the patient during treatment; monitor (112) the data of the clinical parameters and/or anthropometric indicators (102c) of the patient (100) by a health care professional (110) specialist in metabolic syndrome and related disorders, for the assessment of their health status; adjusting or maintaining the indications (113) for the treatment of metabolic syndrome and/or related disorders suffered by the patient or for the prevention of metabolic syndrome and/or related disorders.

In non-limiting embodiments or aspects, the useful method for the measurement, diagnosis, treatment, and management of metabolic syndrome comprising the administration of a drug product to a patient in need thereof, to prevent or reduce the risk of metabolic syndrome and related disorders, by using of electronic devices, the method comprising: through a pharmacy (120), providing (121a) to a patient (100) in need thereof, a pharmaceutical composition with a useful drug product (130) for the prevention, reduction, or management of metabolic syndrome and/or related disorders; through a pharmacy (120), providing (121b) to a patient (100) in need thereof, supplies (140a) or support devices (140b) for the prevention, reduction, or control of metabolic syndrome and/or related disorders; connecting (151) using an interactive user interface (150) one or more devices for measuring clinical parameters and/or anthropometric indicators (140b); measuring (141), recording (142) and storing (143) in the database (103) the collected clinical parameters and/or anthropometric indicators (102c); monitoring (112) the data of the clinical parameters and/or anthropometric indicators (102c) of the patient (100) by a health care professional (110) specialist in metabolic syndrome and related disorders, for the assessment of their health status; adjusting or maintaining the indications (113) for the treatment of metabolic syndrome and/or related disorders suffered by the patient or for the prevention of metabolic syndrome and/or related disorders.

In non-limiting embodiments or aspects, the useful method for the measurement, diagnosis, treatment, and management of metabolic syndrome comprising the administration of a drug product to a patient in need thereof, to prevent or reduce the risk of metabolic syndrome and related disorders, by using electronic devices, the method comprising: through a pharmacy (120), providing (121a) to a patient (100) in need thereof, a pharmaceutical composition with a useful drug product (130) for the prevention, reduction, or control of metabolic syndrome and/or related disorders; connecting (151) using an interactive user interface (150) one or more devices for measuring clinical parameters and/or anthropometric indicators (140b); measuring (141), recording (142) and storing (143) in the database (103) the collected clinical parameters and/or anthropometric indicators (102c); monitoring (112) the data of the clinical parameters and/or anthropometric indicators (102c) of the patient (100) by a health care professional (110) specialist in metabolic syndrome and related disorders, for the assessment of their health status; adjusting or maintaining the indications (113) for the treatment of metabolic syndrome and/or related disorders suffered by the patient or for the prevention of metabolic syndrome and/or related disorders.

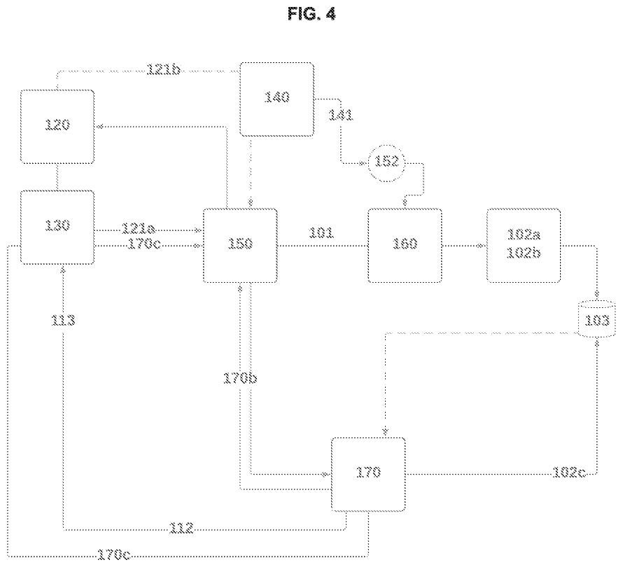

FIG. 4 illustrates non-limiting embodiments or aspects of the system to monitor at least one clinical parameter and/or anthropometric indicator to a patient (100) in need thereof, to prevent or reduce the risk of metabolic syndrome and related disorders. The system includes a device and interface to enter patient identity information and/or body metrics information into a database (160a); a device and interface to collect and store patient identity information and/or body metrics information in a database, for the incorporation into a medical record (160b); means of access for liaison between the patient and a health care professional specialist in metabolic syndrome and related disorders, for assessment of their health status (170); means for indicating to the patient his health status and recording in the medical record (170a); means to instruct the patient on indications for treatment of metabolic syndrome and/or related disorders from which he suffers or for prevention of metabolic syndrome and/or related disorders (170b); means for provide to a patient in need thereof (120), a pharmaceutical composition with a useful drug product for the prevention, reduction or control of metabolic syndrome and/or related disorders (121a); means for providing to a patient in need thereof, supplies or support devices for the prevention, reduction or control of metabolic syndrome and/or related disorders (121b); means to connect by using an interactive user interface to one or more devices for measuring clinical parameters and/or anthropometric indicators (152); a database for recording and storing the collected clinical parameters and/or anthropometric indicators (103); means of monitoring the data of the clinical parameters and/or anthropometric indicators of the patient by a health care professional specialist in metabolic syndrome and related disorders, for the assessment of their health status (112); means of access for the health care professional to adjust or maintain the indications for the treatment of metabolic syndrome and/or related disorders suffered by the patient or for the prevention of metabolic syndrome and/or related disorders (113); optionally, means of access to establish communication between the patient and the health care professional, at established periods of time or at the request of the patient or the health care professional (114).

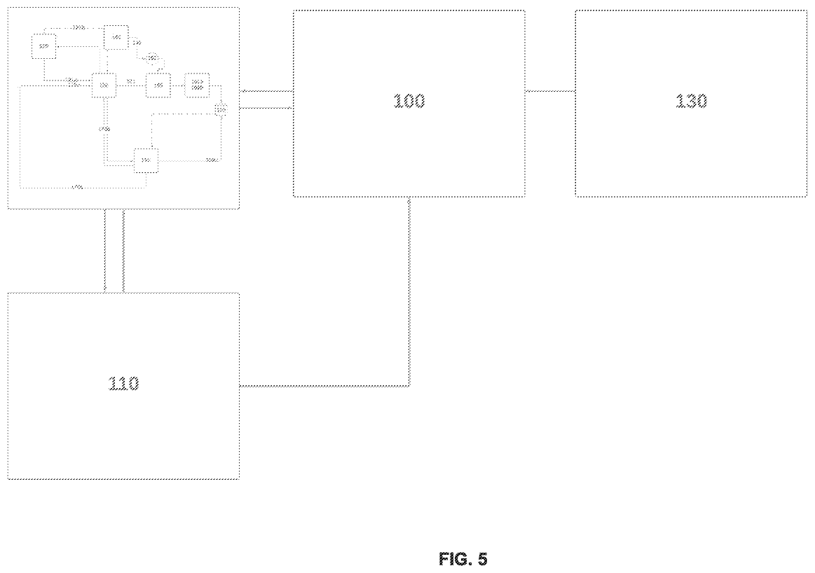

FIG. 5 illustrates non-limiting embodiments or aspects of the useful method for the measurement, diagnosis, treatment, and management of metabolic syndrome comprising the administration of a drug product to a patient in need thereof, to prevent or reduce the risk of metabolic syndrome and related disorders, by using electronic devices, the method comprising: providing a monitoring system (200) for treatment or prevention to the patient and the health care professional comprising at least: a device and interface to enter the patient identity information and/or body metrics information into a database (160a); a device and interface to collect and store patient identity information and/or body metrics information in a database, for incorporation of a medical record (160b): means of access for liaison between the patient and a health care professional specialist in metabolic syndrome and related disorders, for assessment of their health status (170); means to indicate to the patient his health status and recording in the medical record (170a); means to instruct to the patient on indications for treatment of metabolic syndrome and/or related disorders from which he suffers or for prevention of metabolic syndrome and/or related disorders (170b); means to provide to a patient in need thereof (120), a pharmaceutical composition with a useful drug product for the prevention, reduction or control of metabolic syndrome and/or related disorders (121a); means to provide to a patient in need thereof, supplies or support devices for the prevention, reduction or control of metabolic syndrome and/or related disorders (121b); means of connection by using an interactive user interface for one or more devices for measuring clinical parameters and/or anthropometric indicators (152); a database to record and store the collected clinical parameters and/or anthropometric indicators (103); means of monitoring the data of the clinical parameters and/or anthropometric indicators of the patient by a health care professional specialist in metabolic syndrome and related disorders, for the assessment of their health status (112); means of access for the health care professional to adjust or maintain the indications for the treatment of metabolic syndrome and/or related disorders suffered by the patient or for the prevention of metabolic syndrome and/or related disorders (113); optionally, means of access for establishing communication between the patient and the health care professional, at set periods of time or at the request of the patient or the health care professional (114); providing to the patient (100) a pharmaceutical composition with a useful drug product (130) for the prevention, reduction, or control of metabolic syndrome and/or related disorders, from patient identity information and body metrics information or a medical record of the patient, previously stored in a database; monitoring the data of the clinical parameters and/or anthropometric indicators of the patient by a healthcare professional (110) specialist in metabolic syndrome and related disorders, for the assessment of their health status by using an interactive user interface connected to one or more devices for measuring clinical parameters and/or anthropometric indicators.

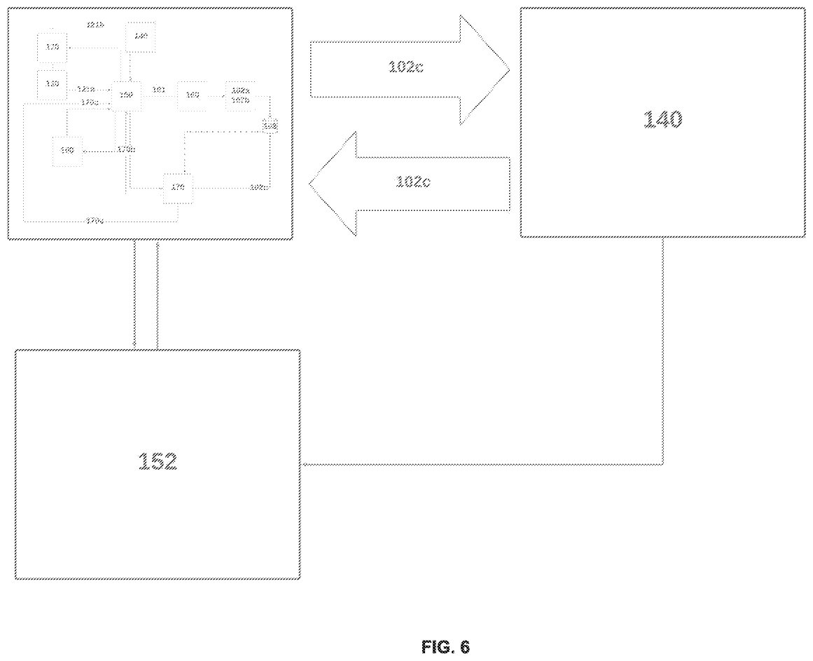

FIG. 6 illustrates non-limiting embodiments or aspects of a system to monitor at least one clinical parameter and/or anthropometric indicator in a patient in need thereof, to prevent or reduce the risk of metabolic syndrome and related disorders, by using electronic devices associated with devices for measuring clinical parameters and/or anthropometric indicators, comprising: a patient monitoring module installed in the electronic device (160a) of the patient; at least one device for measuring clinical parameters and/or anthropometric indicators (140) with connectivity to the electronic device (160a) of the patient; a means of liaison (180) between the electronic device (160a) of the patient and the device for measuring clinical parameters and/or anthropometric indicators (140), wherein the patient monitoring module is configured to (i) collect (101) patient identity information (102a) and/or body metrics information (102c); (ii) (141), record (142) and store (143) in the database (103) the collected clinical parameters and/or anthropometric indicators (102c) of the patient; (iii) store the information in a database (103) for incorporation into a medical record (102b); (iv) link (111) the patient (100) with a health care professional (110) specialist in metabolic syndrome and related disorders, for the assessment of their health status, indicate to the patient their health status (111b), recording in the medical record (102b) and instructing the patient (111c) the indications for the treatment of metabolic syndrome and/or related disorders from which he suffers or for the prevention of metabolic syndrome and/or related disorders; (v) through a pharmacy (120), provide (121a) to a patient (100) in need thereof, a pharmaceutical composition with a useful drug product (130) for the prevention, reduction or control of the metabolic syndrome and/or related disorders, as well as the supplies (140a) or support devices (140b); (vi) establishing a connection (151) with at least one device for measuring clinical parameters and/or anthropometric indicators (140); (vii) measuring (141), recording (142) and storing (143) in the database (103) the clinical parameters and/or anthropometric indicators (102c); (viii) measuring (141), recording (142) and storing (143) in the database (103) clinical parameters and/or anthropometric indicators (102c) obtained from at least one device for measuring clinical parameters and/or anthropometric indicators (140); (vii) allow access (170) to the health care professional specialist in metabolic syndrome and related disorders (110) to the patient medical record for the assessment of their health status and the adjustment or maintenance (113) of the indications for treatment; and optionally, (ix) establishing communication (114) between the patient (100) and the health care professional (110), at established time periods or at the request of the patient or the health care professional.

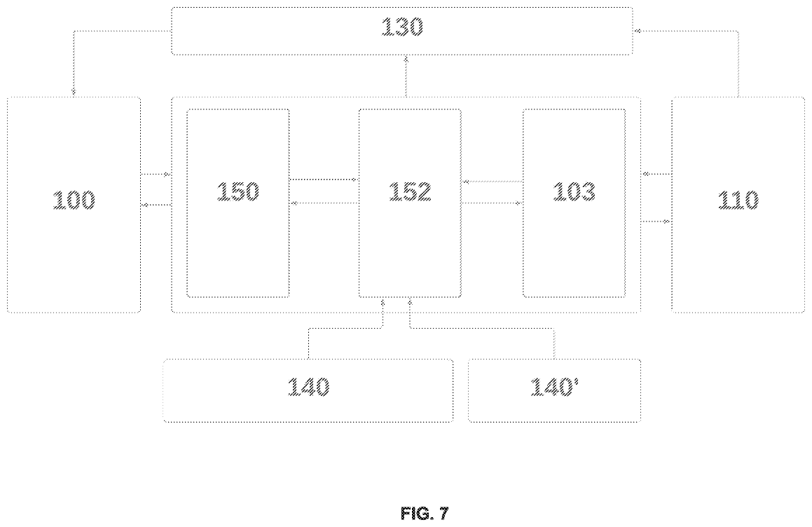

FIG. 7 illustrates non-limiting embodiments or aspects of the connectivity model of the monitoring system connectivity of at least one clinical parameter and/or anthropometric indicator to a patient (100) in need thereof, to prevent or reduce the risk of metabolic syndrome and related disorders, by a health care professional (110); by using electronic devices associated with devices for measuring clinical parameters and/or anthropometric indicators (140), a pharmaceutical composition with a useful drug product (130) for the prevention, reduction or control of metabolic syndrome and/or related disorders comprising: a user interface, a means of liaison, and a database.

The patient, the health care professional, or authorized third parties to access the interface or the information stored in the database may visualize through the interface the clinical parameters and/or anthropometric indicators, as well as the patient information and record. The information is provided to the patient or an authorized third party indicating the standard ranges of the measurement and indicating if there is any risk factor to be addressed; or to show the achieved progress with the treatment, allowing both the patient and the health care professional or authorized third party for monitoring, to carry out immediate actions to achieve the goal set for the treatment.

The device and interface for entering patient identity information and/or body metrics information into a database (160a) and the a device and interface to collect and store patient identity information and/or body metrics information in a database, for incorporation into a medical record (160b), can be any laptop or desktop computer, electronic tablet, smart display, smart screen, smart cell phone, video game console, media player devices, smart watch, smart bracelet, or any device with the ability to enter data either by written text, sound, or in some encoding; and with wired or wireless connectivity.

In non-limiting embodiments or aspects, the link (111) of the patient (100) with a health care professional (110) specialist in metabolic syndrome and related disorders, for the assessment of their health status; is performed by video call, by using electronic devices, prior identification of the patient and the health care professional. Once the link has been made, the medical office is carried out, integrating all the information from the patient file or in situ measurements using the devices for measuring clinical parameters and/or anthropometric indicators; resulting in the indications for treatment by the physician and his dispensing order to provide a pharmaceutical composition with a useful drug product for the prevention, reduction, or control of the metabolic syndrome and/or related disorders.

The identification of the patient and the health care professional can be done through several embodiments, such as password, identification number, username, or biometric identification (fingerprint, retina, or face identification), identification card, among other means of identification.

For its operation, the user interface for both the patient and the health care professional can be connected to one or more screens, printers, speakers and/or other devices. The interface may include notifications, links, buttons, tabs, check boxes, bullets, text fields, drop-down boxes, etc., and may provide various outputs in response to user input, such as text, static images, videos, audio, and animations.

Connection to be selected from a local area network, wireless local area network, tree or ring topography network, wireless internet point of presence systems, ethernet, internet, radio communications, infrared, fiber optic, and 2G, 3G, 4G or 5G data telephony.

The pharmaceutical composition with a useful drug product (130) for the prevention, reduction, or control of metabolic syndrome and/or related disorders, comprises drug products for the prevention, reduction, or control of overweight, obesity, hyperglycemia (type I diabetes or type II diabetes), hypertension, heart disease, dyslipidemia, among other related disorders.

In non-limiting embodiments or aspects, the pharmaceutical composition with a useful drug product (130) for the prevention, reduction, or control of metabolic syndrome and/or related disorders, comprises drug products for the prevention, reduction, or control of overweight and obesity, can be selected from: chemical uncouplers of oxidative phosphorylation, tyrphostin AG17, orlistat, L-carnitine, benzphetamine, phendimetrazine, diethylpropion, liraglutide, naltrexone, phentermine, topiramate, glibenclamide, metformin, acarbose, canagliflozin, dapagliflozin, saxagliptin, dulaglutide, empagliflozin, exenatide, lixisenatide, semaglutide, CBD, sibutramin, hydroxycitric acid, coenzyme q10, chromium picolinate, gamma-linolenic acid, resveratrol, omega-3 fatty acid, antioxidants, vitamins, vitamin B, vitamin C, adrenal glandular extract, pantothenic acid, Curcuma longa, Bayberry bark, Myrica cerifera, Oryza sativa L. indica, Camellia sinensis, Theobroma cacao, Emblica officinalis, Ilex paraguariensis, Cascara sagrada bark, Rhamnus purshiana, Acacia nilotica, persimmon, Blueberry leaf, Grape seed, Cordia salicifolia, Hypericum perforatum, Pueruria thomsonn, Capparis spinosa, Gymnema sylvestre, Elderberry, Sambicus, Citrus aurantium, Coffea canefora, Sygium cumini, Rosmarinus officinalis, Zingiber officinalis, Cassia nomame, Cissus quadrangularis, Phaseolus vulgaris, Licorice Root, Fenugreek, Yohimbe, White willow, Cordyceps sinensis, Ashwaganda, Astragalus, Gingseng, Schisandra, Siberian gingseng, Asian gingseng, Commiphora mukul, Momordica charantia, Garcinia cambogia, Althaea officinalis, Bougainvillea spectabilis, Medicago sativa, Valeriana officinalis, Damiana, Sambucus nigra, Capsicum genus, Allium genus, calcium myristate, magnesium myristate, tetradecylthioacetic acid (TTA), thyroid hormones or their precursors, enhancers of thyroid function, Kreb's cycle metabolites, endogenous uncoupling protein(s), such as UCP-1, UCP-2, UCP-3, PUMP (plant uncoupling mitochondrial protein), or its (their) precursor(s), agonist(s), or enhancers, leucine, valine, isoleucine, glutamine, proline, tyrosine, conjugated linoleic acid, adrenaline secretion enhancers, fatty acids or esters, beta agonists, glucagon, arbutamine, vasopressin, ubiquinone, coenzyme 01, coenzyme Q2, melatonin, fatty acid esters of estrogens, such as oleylestrone, glucomannan, menthol, peppermint essential oil, thyme essential oil, elemental sulfur, and policosanols, insulin or insulin analogues and orally active hypoglycemic agents; acetohexamide, carbutamide, chlorpropamide, glibenclamide, glibornuride, gliclazide, glimepiride, glipizide, tolazamide, and tolbutamide. 2R,3R,4R,5S)-1-(2-hydroxyethyl)-2-(hydroxymethyl)piperidine-3,4,5-triol or Glyset) (1S,2S,3R,4S,5S)-5-(1,3-dihydroxypropan-2-ylamino)-1-(hydroxymethyl)-cyclohexane-1,2,3,4-tetraol), (RS)-5-[4-(2-[methyl(pyridin-2-yl)amino]ethoxy)benzyl]tiazo-lidine-2,4-di-one, (RS)-5-(4-[2-(5-ethylpyridin-2-yl)ethoxy]benzyl)thiazolidine-2,4-dione, 5-[(4-[2-([6-(4-Methoxyphenoxyl)pyrimidin-4-yl]-methylamino)ethoxy]phenyl-)methyl]-1,3-thiazolidine-2,4-dione, (RS)-5-(4-[(6-hydroxy-2,5,7,8-tetramethylchroman-2-yl)methoxy]benzyl)thia-zolidine-2,4-dione. (S)-(+)-2-ethoxy-4-[2-(3-methyl-1-[2-(piperidin-1-yl)phenyl]butylamino)-2--oxoethyl]benzoic acid, 3-phenyl-2-[(4-propan-2-ylcyclohexanecarbonyl)amino]propanoic acid, (2S)-2-benzyl-4-[(3aR,7aS)-octahydro-2H-isoindo1-2-yl]-4-oxobutanoic acid, L-ornithine, L-tyrosine, L-tryptophan, L-phenylalanine, conjugated linoleic acid, gamma-linolenic acid, chromium picolinate, glucose tolerance factor, vanadyl sulfate, Gymnema sylvestre, bromelain, pancreatin, papain, coenzyme Q10, curcumin, barberry, bearberry, Silymarin, Teucrium polium, choline, inositol, human growth hormone, DHEA (dehydroepiandrosterone), caffeine, xanthenes (e.g., fucoxanthin), kola nut, psyllium, yerba mate, guarana, ginseng, medium chain triglycerides, hydroxycitric acid (HCA), kelp, lecithin, dihydroxyacetone, pyruvate, creatine, iodine, niacin, bladderwrack, white bean extract, glucomannan, chitosan, resveratrol, resveratrol derivatives, vitamin D, hCG, capsaicin, chia, hoodia, apple cider vinegar, coconut oil, bitter orange, B vitamin, fluoxetine, chitosan, chromium picolinate, conjugated linoleic acid (CLA), glucomannan, green tea extract, green coffee extract, guar gum, hoodia, 7-keto-DHEA, DHEA, ephedra, bitter orange, turmetic, ginger, root powder, apple cider vinegar, calcium, hesperidin, apantipan gynostemma, extract flaxseed oil, orange peel powder, thiamin, ashwagandha extract, coffee extract, L-theanine, choline bitartrate, red mango extract, white kidney bean extract, c. cinephora robusta extract, acetyl—L carnitine, garcinia, cumbusia extract, coconut oil, forskolin extract, black pepper extract seroxat, citalopram, bupropion, topiramate, ecopipam, naltrexone, peptide YY3-36, leptin, examphetamine, amphetamine, phentermine, mazindol phendimetrazine, diethylpropion, fenfluramine, dexfenfluramine, pharmaceutically acceptable salts and its derivatives, and mixtures thereof.

In non-limiting embodiments or aspects, the pharmaceutical composition with a useful drug product (130) for the prevention, reduction, or control of metabolic syndrome and/or related disorders, comprises drug products for the prevention, reduction, or control of hyperglycemia (type I diabetes or type II diabetes), can be selected from: chemical uncouplers of oxidative phosphorylation, biguanides, imidazolines, alpha-glucosidase inhibitors, meglitinidas, oxadiazolidindiones, thiazolidindiones, insulin sensitizers, sulphonylureas, potassium channel blockers such as nateglinide or BTS-67582, inhibitors of glucagon antagonists DPP-IV (dipeptidyl peptidase-IV), inhibitors of PTPasa (protein tyrosine phosphatase), glucokinase activators, enzymes and metabolites involved in the regulation of gluconeogenesis or glycogenolysis, modulators of glucose uptake, inhibitors GSK-3 (glycogen synthase kinase-3), compounds that modify lipid metabolism such as antihyperlipidemic agents and antilipidemic agents, compounds that reduce food intake and PPAR agonists; for example: tyrphostin AG17, tyrphostin A9, insulin and insulin analogs, tolbutamide, chlorpropamide, tolazamide, glibenclamide, glipizide, glimepiride, glicazide o gliburide, metformin, repaglinide or senaglinide/nateglinide, troglitazone, pioglitazone, rosiglitazone, isaglitazone, darglitazone, englitazone, GI 262570, YM-440, MCC-555, JTT-501, AR-H039242, KRP-297, GW-409544, CRE-16336, AR-H, voglibose, emiglitate, miglitol o acarbose, tolbutamide, glibenclamide, glipizide, glicazide, BTS-67582 o repaglinide, nateglinide, colestiramine, colestipol, clofibrate, gemfibrozil, lovastatine, pravastatine, simvastatine, pharmaceutically acceptable salts and its derivatives, and mixtures thereof.

In non-limiting embodiments or aspects, the pharmaceutical composition with a useful drug product (130) for the prevention, reduction, or control of metabolic syndrome and/or related disorders, comprises drug products for the prevention, reduction, or control of hypertension, can be selected from: alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and [alpha]-blockers such as doxazosin, urapidil, prazosin and terazosin, pharmaceutically acceptable salts and its derivatives, and mixtures thereof.

The chemical uncouplers of oxidative phosphorylation include but are not limited to the following compounds: 4,4-Bis-(4-Hydroxy-3-Nitrophenyl)-Valeric Acid, 4-Methoxy-2-Nitrophenol, 4-Hydroxy-3-Nitroacetophenone, 7-Hydroxy-4-Methyl-8-Nitro-Chromen-2-One, 3-Tert-Butyl-5-Chloro-N-(2-Chloro-4-Nitrophenyl)-2-Hydroxy-6-Methyl-Benzamide, N-1-[4-Cyano-3-(Trifluoromethyl)Phenyl]-3,5-Di(Trifluoromethyl)Benzamide, N-(4-Cyanophenyl)Benzamide, 2′-Chloro-1-Hydroxy-4′-Nitro-2-Naphthanilide, N-(2-Chloro-4-Bromophenyl)-5-Bromosalicylanilide, N-(2-Chloro-4-Nitrophenyl)-3-Tert-Butyl-6-Methylsalicylanilide, (3,5-Di-Tert-Butyl-4-Hydroxybenzyl) Triphenylphosphonium Bromide, 3,5-Di-Tert-Butyl-4-Hydroxybenzyl)Tricyclohexylphosphonium Bromide, (3,5-Di-Tert-Butyl-4-Hydroxybenzyl)Tributylphosphonium Bromide, (3,5-Di-Tert-Buty4-Hydroxybenzyl)Trioctylphosphonium Bromide, 2-Cyano-3-(3,5-Di-Tert-Butyl-4-Hydroxyphenyl)-Acrylic Acid Ethyl Ester, 2-(3,5-Di-Tert-Butyl-4-Hydroxy-Benzylidene)-Malonic Acid Diethyl Ester, 2-Amino-S-[(3,5-Di-Tert-Butyl-4-Hydroxybenzylidone)-Amino]-But-2-Enedinitrile, 2-(3,5-Di-Tert-Butyl-4-Hydroxy-Benzylidene)-Indan-1,3-Dione, 2-[[2-(4-Chlorophenyl)-1H-Indol-3-Yl]Methylene]Malononitrile, 2-(4-Chlorophenyl)-Indole, N-(2,4,5-Trichlorophenyl) Salicylanilide, 2,3-Dimethyl-5-Cyano-7-Ethylindole, 4-Bromo-2-(4-Chlorophenyl)-5-Trifluoromethyl-1H-Pyrrole-3-Carbonitrile, N-(3-Cyano-4-Phenylsulfanyl-Phenyl)-3-Trifluoromethyl-Benzamide, 2,4-Dinitrophenol, Carbonylcyanide p-Trifluoromethoxy-Phenylhydrazone, 2-(5,7-Dimethyl-1H-Indol-3-Ylmethylene)-Malononitrile, 2-(5-Bromo-1H-Indol-3-Ylmethylene)-Malononitrile, 2-((5-Chloro-1H-Indol-3-Yl)Methylene)Malononitrile, 2-((5-Methyl-1H-Indol-3-Yl)Methylene)Malononitrile, 2-((5-Methyl-1H-Indol-3-Yl)Methylene)Malononitrile, 2-(2-Phenyl-3-Indolylmethylene)-Malononitrile, 2-(2-Chloro-1H-Indol-3-Ylmethylene)-Malononitrile, 2-(5-Nitro-1H-Indol-3-Ylmethylene)-Malononitrile, 2-(2-Methyl-5-Nitro-1H-Indol-3-Ylmethylene)-Malononitrile, 3-Bromo-5-Tert-Butyl-N-(2-Chloro-4-Nitro-Phenyl)-6-Hydroxy-2-Methyl-Benzamide, N-(2-Chloro-4-Nitro-Phenyl)-2-Hydroxy-3-Isopropyl-Benzamide, N-(2-Chloro-4-Nitro-Phenyl)-2-Hydroxy-3-Isopropyl-6-Methyl-Benzamide, 3,5-Di-Tert-Butyl-N-(2-Chloro-4-Nitro-Phenyl)-2-Hydroxy-Benzamide, 3-Bromo-N-(2-Chloro-4-Nitro-Phenyl)-6-Hydroxy-5-Isopropyl-2-Methyl-Benzamide, 3-Tert-Butyl-5-Chloro-N-(4-Chloro-3-Trifluoromethyl-Phenyl)-2-Hydroxy-6-Methyl-Benzamide, 3-Tert-Butyl-5-Chloro-N-(4-Cyano-3-Trifluoromethyl-Phenyl)-2-Hydroxy-6-Methyl-Benzamide, 2-Hydroxy-Biphenyl-3-Carboxylic Acid (2-Chloro-4-Nitro-Phenyl)-Amide, 3-Tert-Butyl-N-(2-Chloro-4-Nitro-Phenyl)-2-Hydroxy-5-Methyl-Benzamide, N-(2-Chloro-4-Nitro-Phenyl)-2-Hydroxy-6-Isopropyl-3-Methyl-Benzamide, N-(3,5-Bis-Trifluoromethyl-Phenyl)-3-Tert-Butyl-5-Chloro-2-Hydroxy-6-Methyl-Benzamide, 3-Tert-Butyl-5-Chloro-N-(2-Fluoro-5-Trifluoromethyl-Phenyl)-2-Hydroxy-6-Methyl-Benzamide, 3-Tert-Butyl-5-Chloro-2-Hydroxy-6-Methyl-N-(4-Nitro-3-Trifluoromethyl-Phenyl)-Benzamide, 3-Tert-Butyl-5-Chloro-2-Hydroxy-6-Methyl-N-(4-Nitro-2-Trifluoromethyl-Phenyl)-Benzamide, 3-Bromo-5-Tert-Butyl-N-(2-Chloro-4-Cyano-Phenyl)-6-Hydroxy-2-Methyl-Benzamide, 3-Bromo-5-Tert-Butyl-N-(2-Chloro-5-Trifluoromethyl-Phenyl)-6-Hydroxy-2-Methyl-Benzamide, 3-Bromo-5-Tert-Butyl-N-(2,4-Dichloro-Phenyl)-6-Hydroxy-2-Methyl-Benzamide, 3-Bromo-5-Tert-Butyl-N-(2,4-Dichloro-6-Nitro-Phenyl)-6-Hydroxy-2-Methyl-Benzamide, 3-Bromo-5-Tert-Butyl-N-(2,6-Dichloro-4-Nitro-Phenyl)-6-Hydroxy-2-Methyl-Benzamide, 3-Bromo-5-Tert-Butyl-N-{5-Chloro-4-[(4-Chloro-Phenyl)-Cyano-Methyl]-2-Methyl-Phenyl}-6-Hydroxy-2-Methyl-Benzamide, 3-Bromo-6-Hydroxy-5-Isopropyl-2-Methyl-N-(4-Nitro-2-Trifluoromethyl-Phenyl)-Benzamide, 3-Bromo-5-Tert-Butyl-6-Hydroxy-2-Methyl-N-(4-Nitro-2-Trifluoromethyl-Phenyl)-Benzamide, 3-Tert-Butyl-2-Hydroxy-6-Methyl-N-(4-Nitro-2-Trifluoromethyl-Phenyl)-Benzamide, 3-Bromo-N-(2-Bromo-3,5-Bis-Trifluoromethyl-Phenyl)-5-Tert-Butyl-6-Hydroxy-2-Methylbenzamide, N-(2,5-Bis-Trifluoromethyl-Phenyl)-3-Bromo-5-Tert-Butyl-6-Hydroxy-2-Methyl-Benzamide, 3-Bromo-5-Tert-Butyl-N-(2,4-Dichloro-6-Trifluoromethyl-Phenyl)-6-Hydroxy-2-Methylbenzamide, 3-Bromo-5-Tert-Butyl-6-Hydroxy-N-(4-Isopropyl-2-Trifluoromethyl-Phenyl)-2-Methyl-Benzamide, N-(3,5-Bis-Trifluoromethyl-Phenyl)-3-Fluoro-5-Trifluoromethyl-Benzamide, 3-Fluoro-N-(4-Nitro-3-Trifluoromethyl-Phenyl)-5-Trifluoromethyl-Benzamide, N-(3,5-Bis-Trifluoromethyl-Phenyl)-3-Fluoro-4-Trifluoromethyl-Benzamide, 4-Fluoro-N-(4-Nitro-3-Trifluoromethyl-Phenyl)-3-Trifluoromethyl-Benzamide, 3-(3,5-Di-Tert-Butyl-4-Hydroxy-Phenyl-2-(2,2-Dimethyl-Propionyl)-Acrylonitrile, 2-Acetyl-3-(3,5-Di-Tert-Butyl-4-Hydroxy-Phenyl)-Acrylic Acid Ethyl Ester, 2-(3,5-Dimethyl-4-Hydroxy-Benzylidene)-Malononitrile, 2-(3,5-Dimethyl-4-Hydroxy-Benzylidene)-Malononitrile, 2,6-Di-Tert-Butyl-4-Nitro-Phenol, 2-Tert-Butyl-4,6-Dinitro-Phenol, 3-(3,5-Di-Tert-Butyl-4-Hydroxy-Phenyl)-2- Pyridin-2-Yl-Acrylonitrile, 2-[1-(3,5-Di-Tert-Butyl-4-Hydroxy-Phenyl)-Ethylidene]-Malononitrile, 3-(3,5-Di-Tertbutyl-4-Hydroxybenzylidene)-2-(Diethylphosphonate)-Propenenitrile, 3-(3,5-Di-Tert-Butyl-4-Hydroxy-Phenyl)-2-(4-Nitro-Phenyl)-Acrylonitrile, 3-(3,5-Di-Tert-Butyl-4-Hydroxy-Phenyl)-2-Pyridin-4-Yl-Acrylonitrile, 2-(3,5-Bis-Trifluoromethyl-Phenyl)-3-(3 ,5-Di-Tert-Butyl-4-Hydroxy-Phenyl)-Acrylonitrile, 3-(3,5-Di-Tert-Butyl-4-Hydroxy-Phenyl)-2-(4-Trifluoromethoxy-Phenyl)-Acrylonitrile, 3-(3,5-Di-Tert-Butyl-4-Hydroxy-Phenyl)-2-(4-Trifluoromethyl-Phenyl)-Acrylonitrile, 2-Cyano-3-(3,5-Di-Tert-Butyl-4-Hydroxy-Phenyl)-But-2-Enoic Acid Ethyl Ester, N-(4-Chloro-Phenyl)-2-Cyano-3-(3,5-Di-Tert-Butyl-4-Hydroxy-Phenyl)-Acrylamide, (E)-3-(3,5-Di-Tert-Butyl-4-Hydroxy-Phenyl)-2-Methanesulfonyl-Acrylonitrile, (E)-2-(4-Chloro-Benzenesulfonyl)-3-(3,5-Di-Tert-Butyl-4-Hydroxy-Phenyl)-Acrylonitrile, (E)-3-(3,5-Di-Tert-Butyl-4-HydroxyPhenyl)-2-(4-Fluoro-Benzenesulfonyl)-Acrylonitrile, (E)-2-B enzene sulfonyl-3-(3,5-Di-Tert-Butyl-4-Hydroxy-Phenyl)-Acrylonitrile, (E)-3-(3,5-Di-Tert-Butyl-4-Hydroxy-Phenyl)-2-(Propane-2-Sulfonyl)-Acrylonitrile, 3-(3,5-Di-Tert-Butyl-4-Hydroxy-Phenyl)-2-(2,5-Dichloro-Benzenesulfonyl)-Acrylonitrile, 3-(3,5-Di-Tert-Butyl-4-Hydroxy-Phenyl)-2-(2,4-Dichloro-Benzenesulfonyl)-Acrylonitrile, 3-(3,5-Di-Tert-Butyl-4-Hydroxy-Phenyl)-2-(Hexane-1-Sulfonyl)-Acrylonitrile, 2-(4-Bromo-Benzenesulfonyl)-3-(3,5-Di-Tert-Butyl-4-Hydroxy-Phenyl)-Acrylonitrile, 2-(3-Bromo-Benzenesulfonyl)-3-(3,5-Di-Tert-Butyl-4-Hydroxy-Phenyl)-Acrylonitrile, 5-Bromo-3-Tert-Butyl-N-(2-Chloro-4-Cyanophenyl)-2-Hydroxybenzamide, 5-Bromo-3-Tert-Butyl-N-(4-Cyanophenyl)-2-Hydroxybenzamide, 3-Bromo-5-Tert-Butyl-6-Hydroxy-2-Methyl-N-(2-Trifluoromethylphenyl)Benzamide, 2-(2-Bromo-1H-Indo1-3-Ylmethylene)Malononitrile, 2-(7-Bromo-2-Methyl-1H-Indol-3-Ylmethylene)Malononitrile, 2-(5-Bromo-2-Methyl-1H-Indol-3-Ylmethylene) Malononitrile, pharmaceutically acceptable salts and its derivatives, and mixtures thereof.

Clinical parameters and/or anthropometric indicators can be selected from: weight, body mass index, % fat, height, muscle, water, visceral fat, bone, metabolism, protein, degree of obesity, body age, fat-free weight, oxygenation, body oxygenation quantified in urine, vitamins and minerals, blood glucose, blood pressure, LDL cholesterol, HDL cholesterol, triglycerides, urine glucose, step counter, heart rate, fasting glucose, post-load glucose, total cholesterol, total bilirubin, alanine transaminase, aspartate aminotransferase and/or body temperature.

The device for measuring clinical parameters and/or anthropometric indicators (140) are selected, but not limited to: scale, ORP, Vitamin stick, glucometer, oximeter, smart watch, smart bracelet, smart clothing, patches, tattoos, implants, hearing aids, helmets, electrodes, chest band, pedometer and/or medical equipment and devices for quantification of biochemical parameters.

Whereas particular embodiments of this invention have been described above for purposes of illustration, it will be evident to those skilled in the art that numerous variations of the details of the present invention may be made without departing from the invention as defined in the appended claims.

What is claimed is:

1. A method for measurement, diagnosis, treatment, and management of metabolic syndrome comprising the administration of a drug product to a patient (100) in need thereof, to prevent or reduce the risk of metabolic syndrome and related disorders, by using electronic devices, the method comprising:
(a) collecting (101) and storing patient identity information (102a) and/or information of body metrics, clinical parameters, and/or anthropometric indicators (102c) in a database (103) for incorporation of a medical record (102b);
(b) linking (111) the patient (100) with a health care professional (110) specialist in metabolic syndrome and related disorders for the assessment of the patient's health status;
(c) indicating to the patient his health status (111b) and recording in the medical record (102b);
(d) instructing the patient (111c) the indications for the treatment of metabolic syndrome and/or related disorders that the patient suffers or for the prevention of metabolic syndrome and/or related disorders;
(e) through a pharmacy (120), providing (121a) to a patient (100) in need thereof, a pharmaceutical composition with a useful drug product (130) for the prevention, reduction, or control of metabolic syndrome and/or related disorders;
(f) through a pharmacy (120), providing (121b) to a patient (100) in need thereof, supplies (140a) or measuring devices (140) for the prevention, reduction, or control of metabolic syndrome and/or related disorders;
(g) connecting (151), by using an interactive user interface (150), one or more devices for measuring clinical parameters and/or anthropometric indicators (140b);
(h) measuring (141), recording (142), and storing (143) in the database (103) the collected clinical parameters and/or anthropometric indicators (102c);
(i) monitoring (112) the data of the clinical parameters and/or anthropometric indicators (102c) of the patient (100) by a health care professional (110) specialist in metabolic syndrome and related disorders for the assessment of their health status;
(j) adjusting or maintaining the indications (113) for the treatment of metabolic syndrome and/or related disorders suffered by the patient or for the prevention of metabolic syndrome and/or related disorders; and
(k) optionally, assisting (114) the patient (100) by the health care professional (110), at established periods of time or at the request of the patient or the health care professional.

2. A method for measurement, diagnosis, treatment, and management of metabolic syndrome comprising the administration of a drug product to a patient in need thereof to prevent or reduce the risk of metabolic syndrome and related disorders, by using electronic devices, the method comprising:
(a) providing (121a) to the patient (100) a pharmaceutical composition with a useful drug product (130) for the prevention, reduction, or control of metabolic syndrome and/or related disorders from patient identity information (102a) and body metrics information (102c) and/or a medical record (102b) of the patient previously stored in a database (103); and
(b) monitoring (112) data of clinical parameters and/or anthropometric indicators of the patient by a health care professional specialist (110) in metabolic syndrome and related disorders for assessment of their health status by using an interactive user interface (150) connected with one or more devices for measuring clinical parameters and/or anthropometric indicators, wherein an interactive user interface (150) records (142) and stores (143) the clinical parameters and/or anthropometric indicators.

3. A method for measurement, diagnosis, treatment, and management of metabolic syndrome comprising administration of a drug product to a patient undergoing treatment prevention, reduction, or control of metabolic syndrome and/or related disorders by using electronic devices, the method comprising:
(a) collecting (101) and storing patient identity information (102a) and/or information of body metrics, clinical parameters, and/or anthropometric indicators (102c) in a database (103) for the incorporation of a medical record (102b);
(b) through a pharmacy (120), providing (121a) to a patient (100) in need thereof, a pharmaceutical composition with a useful drug product (130) for the prevention, reduction, or control of metabolic syndrome and/or related disorders, from the patient identity information (102a) and the patient clinical parameters and/or anthropometric indicators information (102c) and/or a medical record (102b) of the patient previously stored in a database;
(c) measuring (141), recording (142), and storing (143) in the database (103) the collected clinical parameters and/or anthropometric indicators (102c) of the patient during treatment;
(d) monitoring (112) the data of the clinical parameters and/or anthropometric indicators (102c) of the patient (100) by a health care professional (110) specialist in metabolic syndrome and related disorders, for the assessment of the patient's health status; and
(e) adjusting or maintaining indications (113) for the treatment of metabolic syndrome and/or related disorders suffered by the patient or for the prevention of metabolic syndrome and/or related disorders.

4. A method for measurement, diagnosis, treatment, and management of metabolic syndrome comprising administration of a drug product to a patient in need thereof to prevent or reduce a risk of metabolic syndrome and related disorders by using electronic devices, the method comprising:
(a) linking (111) the patient (100) with a health care professional (110) specialist in metabolic syndrome and related disorders, for the assessment of the patient's health status;
(b) indicating to the patient a health status (111b) and recording in a medical record (102b);
(c) instructing the patient (111c) indications for the treatment of metabolic syndrome and/or related disorders that the patient suffers or for prevention of metabolic syndrome and/or related disorders;
(d) through a pharmacy (120), providing (121a) to the patient (100) in need thereof, a pharmaceutical composition with a useful drug product (130) for the prevention, reduction, or control of metabolic syndrome and/or related disorders;
(e) recording (142) and storing (143) in the database (103) body metrics, clinical parameters, and/or anthropometric indicators collected (102c) from the patient during treatment;
(f) monitoring (112) the data of the clinical parameters and/or anthropometric indicators of the patient (102c) (100) by the health care professional (110) specialist in metabolic syndrome and related disorders for assessment of the patient's health status; and
(g) adjusting or maintaining indications (113) for the treatment of metabolic syndrome and/or related disorders suffered by the patient or for the prevention of metabolic syndrome and/or related disorders.

5. A method for measurement, diagnosis, treatment, and management of metabolic syndrome comprising the administration of a drug product to a patient in need thereof to prevent or reduce the risk of metabolic syndrome and related disorders by using electronic devices, the method comprising:
(a) through a pharmacy (120), providing (121a) to the patient (100) in need thereof, a pharmaceutical composition with a useful drug product (130) for the prevention, reduction, or control of metabolic syndrome and/or related disorders;
(b) through the pharmacy (120), providing (121b) to the patient (100) in need thereof supplies (140a) or supportive devices (140b) for the prevention, reduction, or control of metabolic syndrome and/or related disorders;
(c) connecting (151) by using an interactive user interface (150) one or more devices for measuring clinical parameters and/or anthropometric indicators (140b);
(d) measuring (141), recording (142). and storing (143) in the database (103) the collected clinical parameters and/or anthropometric indicators (102c);
(e) monitoring (112) the data (100) clinical parameters and/or anthropometric indicators of the patient (102c) by the health care professional (110) specialist in metabolic syndrome and related disorders for assessment of their health status; and
(f) adjusting or maintaining indications (113) for the treatment of metabolic syndrome and/or related disorders suffered by the patient or for the prevention of metabolic syndrome and/or related disorders.

6. A method for measurement, diagnosis, treatment, and management of metabolic syndrome comprising the administration of a drug to a patient in need thereof to prevent or reduce the risk of metabolic syndrome and related disorders by using electronic devices, the method comprising:
(a) through a pharmacy (120), providing (121a) to the patient (100) in need thereof, a pharmaceutical composition with a useful drug product (130) for the prevention, reduction, or control of metabolic syndrome and/or related disorders;
(b) connecting (151) by using an interactive user interface (150) one or more devices for measuring clinical parameters and/or anthropometric indicators (140b);
(c) measuring (141), recording (142), and storing (143) in the database (103) the collected clinical parameters and/or anthropometric indicators (102c);
(d) monitoring (112) the data (100) clinical parameter and/or anthropometric indicators of the patient (102c) by a health care professional (110) specialist in metabolic syndrome and related disorders for assessment of their health status; and
(e) adjusting or maintaining the indications (113) for the treatment of metabolic syndrome and/or related disorders suffered by the patient or for the prevention of metabolic syndrome and/or related disorders.

7. A system for monitoring at least one clinical parameter and/or anthropometric indicator to a patient (100) in need thereof, to prevent or reduce the risk of metabolic syndrome and related disorders by using an electronic device associated with devices for measuring clinical parameters and/or anthropometric indicators, the system comprising:
(a) a patient monitoring module installed in the electronic device (160a) of the patient;
(b) at least one device for measuring clinical parameters and/or anthropometric indicators (140) with connectivity to the electronic device (160a) of the patient; and
(c) a means of liaison (180) between the electronic device (160a) of the patient and the device for measuring clinical parameters and/or anthropometric indicators (140);
wherein the patient monitoring module is configured to:
(i) collect (101) patient identity information (102a) and/or body metrics information (102c);
(ii) record (142) and store (143) in a database (103) the collected clinical parameters and/or anthropometric indicators (102c) of the patient;
(iii) store the information in the database (103) for incorporation into a medical record (102b);
(iv) link (111) the patient (100) with a health care professional (110) specialist in metabolic syndrome and related disorders for the assessment of the patient's health status, indicating to the patient a health status (111b), recording in the medical record (102b), and instructing the patient (111c) indications for the treatment of metabolic syndrome and/or related disorders from which the patient suffers or for the prevention of metabolic syndrome and/or related disorders;
(v) through a pharmacy (120), provide (121a) to the patient (100) in need thereof, a pharmaceutical composition with a useful drug product (130) for the prevention, reduction, or control of the metabolic syndrome and/or related disorders, and supplies (140a) or support devices (140b);
(vi) to establish a connection (151) with at least one device for measuring clinical parameters and/or anthropometric indicators (140);
(vii) measure (141), record (142) and store (143) in the database (103) the clinical parameters and/or anthropometric indicators (102c) obtained from at least one device for measuring clinical parameters and/or anthropometric indicators (140);
(viii) allow access (170) to the health care professional specialist in metabolic syndrome and related disorders (110) to the medical record for the assessment of the patient's health status and the adjustment or maintenance (113) of the indications for treatment; and
(ix) optionally, establishing communication (114) between the patient (100) and the health care professional (110) at established periods of time or upon request of the patient or the health care professional.

8. A system for monitoring at least one clinical parameter and/or anthropometric indicator to a patient (100) in need thereof, to prevent or reduce the risk of metabolic syndrome and related disorders by using electronic devices, the system comprising:
(a) a device and interface for entering patient identity information and/or body metrics information into a database (160a);
(b) a device and interface to collect and store the patient identity information and/or body metrics information in the database for incorporation of a medical record (160b);
(c) means of access for liaison between the patient and a health care professional specialist in metabolic syndrome and related disorders for assessment of the patient's health status (170);
(d) means to indicate the patient the patient's health status and record in the medical record (170a);
(e) means to instruct the patient on the indications for the treatment of metabolic syndrome and/or related disorders that the patient suffers or for the prevention of metabolic syndrome and/or related disorders (170b);
(f) means to provide to the patient in need thereof (120) a pharmaceutical composition containing a useful drug product for the prevention, reduction, or control of metabolic syndrome and/or related disorders (121a);
(g) means to provide to the patient in need thereof supplies or supportive devices for the prevention, reduction, or control of metabolic syndrome and/or related disorders (121b);
(h) means of connection by using an interactive user interface for one or more devices for measuring clinical parameters and/or anthropometric indicators (152);
(i) a database for recording and storing the collected clinical parameters and/or anthropometric indicators (103);
(j) means of monitoring the data of the clinical parameters and/or anthropometric indicators of the patient by the health care professional specialist in metabolic syndrome and related disorders for the evaluation of their health status (112);
(k) means of access for the health care professional to adjust or maintain the indications for the treatment of metabolic syndrome and/or related disorders suffered by the patient or for the prevention of metabolic syndrome and/or disorders (113); and
(l) optionally, means of access to establish communication between the patient and the health care professional at set periods of time or at the request of the patient or the health care professional (114).

9. A method for measurement, diagnosis, treatment, and management of metabolic syndrome comprising the administration of a drug product to a patient in need thereof to prevent or reduce the risk of metabolic syndrome and related disorders by using electronic devices, the method comprising:
(a) providing a monitoring system (200) for treatment or prevention to the patient and a health care professional comprising at least: a device and interface to enter patient identity information and/or body metrics information into a database (160a); a device and interface to collect and store the patient identity information and/or body metrics information in a database for incorporation of a medical record (160b); means of access for liaison between the patient and a health care professional specialist in metabolic syndrome and related disorders for assessment of the patient's health status (170); means to indicate to the patient the patient's health status and record in a medical record (170a); means to instruct the patient on indications for the treatment of metabolic syndrome and/or related disorders that the patient suffers or for the prevention of metabolic syndrome and/or related disorders (170b); means to provide to the patient in need thereof (120) a pharmaceutical composition with a useful drug product for the prevention, reduction or control of metabolic syndrome and/or related disorders (121a); means to provide to the patient in need thereof supplies or supportive devices for the prevention, reduction, or control of metabolic syndrome and/or related disorders (121b); means of connection by using an interactive user interface for one or more devices for measuring clinical parameters and/or anthropometric indicators (152); a database for recording and storing the collected clinical parameters and/or anthropometric indicators (103); means of monitoring the data of the clinical parameters and/or anthropometric indicators of the patient by a health care professional specialist in metabolic syndrome and related disorders for the evaluation of their health status (112); means of access for the health care professional to adjust or maintain the indications for the treatment of metabolic syndrome and/or related disorders suffered by the patient or for the prevention of metabolic syndrome and/or related disorders (113); optionally, means of access to establish communication between the patient and the health care professional at established time periods or at the request of the patient or the health care professional (114);
(b) providing the patient (100) with a pharmaceutical composition with a useful drug product (130) for the prevention, reduction, or control of metabolic syndrome and/or related disorders from the patient identity information and body metrics information or the medical record of the patient previously stored in a database; and
(c) monitoring the data of clinical parameters and/or anthropometric indicators of the patient by the healthcare professional (110) specialist in metabolic syndrome and related disorders for the assessment of their health status by using an interactive user interface connected to one or more devices for measuring clinical parameters and/or anthropometric indicators.

10. The method for the measurement, diagnosis, treatment, and management of metabolic syndrome, comprising the administration of a drug product to a patient (100) in need thereof to prevent or reduce the risk of metabolic syndrome and related disorders by using electronic devices, according to any one of claims 1 to 6, wherein the body metrics, clinical parameters, and/or anthropometric indicators are selected from the group consisting of: weight, body mass index, % fat, height, muscle, water, visceral fat, bone, metabolism, protein, degree of obesity, body age, fat-free weight, oxygenation, body oxygenation quantified in urine, vitamins and minerals, blood glucose, blood pressure, LDL cholesterol, HDL cholesterol, triglycerides, urine glucose, step counter, heart rate, fasting glucose, post-load glucose, total cholesterol, total bilirubin, alanine transaminase, aspartate aminotransferase, body temperature, and any combination thereof.

11. The method for the measurement, diagnosis, treatment, and management of metabolic syndrome, comprising the administration of a drug product to the patient (100) in need thereof to prevent or reduce the risk of metabolic syndrome and related disorders by using electronic devices, according to any one of claims 1 to 6, wherein the measuring devices (140) are selected from the group consisting of: scale, ORP, Vitamin stick, glucometer, oximeter, smart watch, smart bracelet, smart clothing, patches, tattoos, implants, hearing aids, helmets, electrodes, chest band, pedometer, medical equipment for quantification of biochemical parameters, and any combination thereof.

12. The method for the measurement, diagnosis, treatment, and management of metabolic syndrome comprising the administration of a drug to the patient (100) in need thereof to prevent or reduce the risk of metabolic syndrome and related disorders, by using electronic devices, according to any one of claims 1 to 6, wherein the pharmaceutical composition with a drug product (130) useful for the prevention, reduction, or control of metabolic syndrome and/or related disorders is selected from the group consisting of: chemical uncouplers of oxidative phosphorylation, tyrphostin AG17, orlistat, L-carnitine, benzphetamine, phendimetrazine, diethylpropion, liraglutide, naltrexone, phentermine, topiramate, glibenclamide, metformin, acarbose, canagliflozin, dapagliflozin, saxagliptin, dulaglutide, empagliflozin, exenatide, lixisenatide, semaglutide, CBD, sibutramin, hydroxycitric acid, coenzyme q10, chromium picolinate, gamma-linolenic acid, resveratrol, omega-3 fatty acid, antioxidants, vitamins, vitamin B, vitamin C, adrenal glandular extract, pantothenic acid, Curcuma longa, Bayberry bark, Myrica cerifera, Oryza sativa L. indica, Camellia sinensis, Theobroma cacao, Emblica officinalis, Ilex paraguariensis, Cascara sagrada bark, Rhamnus purshiana, Acacia nilotica, persimmon, Blueberry leaf, Grape seed, Cordia salicifolia, Hypericum perforatum, Pueruria thomsonn, Capparis spinosa, Gymnema sylvestre, Elderberry, Sambicus, Citrus aurantium, Coffea canefora, Sygium cumini, Rosmarinus officinalis, Zingiber officinalis, Cassia nomame, Cissus quadrangularis, Phaseolus vulgaris, Licorice Root, Fenugreek, Yohimbe, White willow, Cordyceps sinensis, Ashwaganda, Astragalus, Gingseng, Schisandra, Siberian gingseng, Asian gingseng, Commiphora mukul, Momordica charantia, Garcinia cambogia, Althaea officinalis, Bougainvillea spectabilis, Medicago sativa, Valeriana officinalis, Damiana, Sambucus nigra, Capsicum genus, Allium genus, calcium myristate, magnesium myristate, tetradecylthioacetic acid (TTA), thyroid hormones or their precursors, enhancers of thyroid function, Kreb's cycle metabolites, endogenous uncoupling protein(s) or its (their) precursor(s), agonist(s), or enhancers, leucine, valine, isoleucine, glutamine, proline, tyrosine, conjugated linoleic acid, adrenaline secretion enhancers, fatty acids or esters, beta agonists, glucagon, arbutamine, vasopressin, ubiquinone, coenzyme 01, coenzyme Q2, melatonin, fatty acid esters of estrogens, insulin or insulin analogues and orally active hypoglycemic agents; acetohexamide, carbutamide, chlorpropamide, glibenclamide, glibornuride, gliclazide, glimepiride, glipizide, tolazamide, and tolbutamide., 2R,3R,4R,5S)-1-(2-hydroxyethyl)-2-(hydroxymethyl)piperidine-3,4,5-triol or Glyset) (1S,2S,3R,4S,5S)-5-(1,3-dihydroxypropan-2-ylamino)-1-(hydroxymethyl)-cyclohexane-1,2,3,4-tetraol), (RS)-5-[4-(2-[methyl(pyridin-2-yl)amino]ethoxy)benzyl]tiazo-lidine-2,4-di-one, (RS)-5-(4-[2-(5-ethylpyridin-2-yl)ethoxy]benzyl)thiazolidine-2,4-dione, 5-[(4-[2-([6-(4-Methoxyphenoxyl)pyrimidin-4-yl]-methylamino)ethoxy]phenyl-)methyl]-1,3-thiazolidine-2,4-dione, (RS)-5-(4-[(6-hydroxy-2,5,7,8-tetramethylchroman-2-yl)methoxy]benzyl)thia-zolidine-2,4-dione. (S)-(+)-2-ethoxy-4-[2-(3-methyl-1-[2-(piperidin-1-yl)phenyl]butylamino)-2--oxoethyl]benzoic acid, 3-phenyl-2-[(4-propan-2-ylcyclohexanecarbonyl)amino]propanoic acid, (2S)-2-benzyl-4-[(3aR,7aS)-octahydro-2H-isoindo1-2-yl]-4-oxobutanoic acid, L-ornithine, L-tyrosine, L-tryptophan, L-phenylalanine, conjugated linoleic acid, gamma-linolenic acid, chromium picolinate, glucose tolerance factor, vanadyl sulfate, Gymnema sylvestre, bromelain, pancreatin, papain, coenzyme Q10, curcumin, barberry, bearberry, Silymarin, Teucrium polium, choline, inositol, human growth hormone, DHEA (dehydroepiandrosterone), caffeine, xanthenes (e.g., fucoxanthin), kola nut, psyllium, yerba mate, guarana, ginseng, medium chain triglycerides, hydroxycitric acid (HCA), kelp, lecithin, dihydroxyacetone, pyruvate, creatine, iodine, niacin, bladderwrack, white bean extract, glucomannan, chitosan, resveratrol, resveratrol derivatives, vitamin D, hCG, capsaicin, chia, hoodia, apple cider vinegar, coconut oil, bitter orange, B vitamin, fluoxetine, chitosan, chromium picolinate, conjugated linoleic acid (CLA), glucomannan, green tea extract, green coffee extract, guar gum, hoodia, 7-keto-DHEA, DHEA, ephedra, bitter orange, turmetic, ginger, root powder, apple cider vinegar, calcium, hesperidin, apantipan gynostemma, extract flaxseed oil, orange peel powder, thiamin, ashwagandha extract, coffee extract, L-theanine, choline bitartrate, red mango extract, white kidney bean extract, c. cinephora robusta extract, acetyl—L carnitine, garcinia, cumbusia extract, coconut oil, forskolin extract, black pepper extract seroxat, citalopram, bupropion, topiramate, ecopipam, naltrexone, peptide YY3-36, leptin, examphetamine, amphetamine, phentermine, mazindol phendimetrazine, diethylpropion, fenfluramine, dexfenfluramine, pharmaceutically acceptable salts and its derivatives, and mixtures thereof.

13. The method for the measurement, diagnosis, treatment, and management of metabolic syndrome comprising the administration of a drug product to the patient (100) in need thereof to prevent or reduce the risk of metabolic syndrome and related disorders using electronic devices according to claim 12, wherein the oxidative phosphorylation chemical uncouplers are selected from the group consisting of: 4,4-Bis-(4-Hydroxy-3-Nitrophenyl)-Valeric Acid, 4-Methoxy-2-Nitrophenol, 4-Hydroxy-3-Nitro acetophenone, 7-Hydroxy-4-Methyl-8-Nitro-Chromen-2-One, 3-Tert-Butyl-5-Chloro-N-(2-Chloro-4-Nitrophenyl)-2-Hydroxy-6-Methyl-Benzamide, N-1-[4-Cyano-3-(Trifluoromethyl)Phenyl]-3,5-Di(Trifluoromethyl)Benzamide, N-(4-Cyanophenyl)Benzamide, 2′-Chloro-1-Hydroxy-4′-Nitro-2-Naphthanilide, N-(2-Chloro-4-Bromophenyl)-5-Bromosalicylanilide, N-(2-Chloro-4-Nitrophenyl)-3-Tert-Butyl-6-Methylsalicylanilide, (3,5-Di-Tert-Butyl-4-Hydroxybenzyl) Triphenylphosphonium Bromide, 3,5-Di-Tert-Butyl-4-Hydroxybenzyl)Tricyclohexylphosphonium Bromide, (3,5-Di-Tert-Butyl-4-Hydroxybenzyl)Tributylphosphonium Bromide, (3,5-Di-Tert-Buty-4-Hydroxybenzyl)Trioctylphosphonium Bromide, 2-Cyano-3-(3,5-Di-Tert-Butyl-4-Hydroxyphenyl)-Acrylic Acid Ethyl Ester, 2-(3,5-Di-Tert-Butyl-4-Hydroxy-Benzylidene)-Malonic Acid Diethyl Ester, 2-Amino-S-[(3,5-Di-Tert-Butyl-4- Hydroxybenzylidone)-Amino]-But-2-Enedinitrile, 2-(3,5-Di-Tert-Butyl-4-Hydroxy-Benzylidene)-Indan-1,3-Dione, 2-[[2-(4-Chlorophenyl)-1H-Indol-3-Yl]Methylene]Malononitrile, 2-(4-Chlorophenyl)-Indole, N-(2,4,5-Trichlorophenyl) Salicylanilide, 2,3-Dimethyl-5-Cyano-7-Ethylindole, 4-Bromo-2-(4-Chlorophenyl)-5-Trifluoromethyl-1H-Pyrrole-3-Carbonitrile, N-(3-Cyano-4-Phenylsulfanyl-Phenyl)-3-Trifluoromethyl-Benzamide, 2,4-Dinitrophenol, Carbonylcyanide p-Trifluoromethoxy-Phenylhydrazone, 2-(5,7-Dimethyl-1H-Indol-3-Ylmethylene)-Malononitrile, 2-(5-Bromo-1H-Indol-3-Ylmethylene)-Malononitrile, 2-((5-Chloro-1H-Indol-3-Yl)Methylene)Malononitrile, 2-((5-Methyl-1H-Indol-3-Yl)Methylene)Malononitrile, 2-((5-Methyl-1H-Indol-3-Yl)Methylene)Malononitrile, 2-(2-Phenyl-3-Indolylmethylene)-Malononitrile, 2-(2-Chloro-1H-Indol-3-Ylmethylene)-Malononitrile, 2-(5-Nitro-1H-Indol-3-Ylmethylene)-Malononitrile, 2-(2-Methyl-5-Nitro-1H-Indol-3-Ylmethylene)-Malononitrile, 3-Bromo-5-Tert-Butyl-N-(2-Chloro-4-Nitro-Phenyl)-6-Hydroxy-2-Methyl-Benzamide, N-(2-Chloro-4-Nitro-Phenyl)-2-Hydroxy-3-Isopropyl-Benzamide, N-(2-Chloro-4-Nitro-Phenyl)-2-Hydroxy-3-Isopropyl-6-Methyl-Benzamide, 3,5-Di-Tert-Butyl-N-(2-Chloro-4-Nitro-Phenyl)-2-Hydroxy-Benzamide, 3-Bromo-N-(2-Chloro-4-Nitro-Phenyl)-6-Hydroxy-5-Isopropyl-2-Methyl-Benzamide, 3-Tert-Butyl-5-Chloro-N-(4-Chloro-3-Trifluoromethyl-Phenyl)-2-Hydroxy-6-Methyl-Benzamide, 3-Tert-Butyl-5-Chloro-N-(4-Cyano-3-Trifluoromethyl-Phenyl)-2-Hydroxy-6-Methyl-Benzamide, 2-Hydroxy-Biphenyl-3-Carboxylic Acid (2-Chloro-4-Nitro-Phenyl)-Amide, 3-Tert-Butyl-N-(2-Chloro-4-Nitro-Phenyl)-2-Hydroxy-5-Methyl-Benzamide, N-(2-Chloro-4-Nitro-Phenyl)-2-Hydroxy-6-Isopropyl-3-Methyl-Benzamide, N-(3,5-Bis-Trifluoromethyl-Phenyl)-3-Tert-Butyl-5-Chloro-2-Hydroxy-6-Methyl-Benzamide, 3-Tert-Butyl-5-Chloro-N-(2-Fluoro-5-Trifluoromethyl-Phenyl)-2-Hydroxy-6-Methyl-Benzamide, 3-Tert-Butyl-5-Chloro-2-Hydroxy-6-Methyl-N-(4-Nitro-3-Trifluoromethyl-Phenyl)-Benzamide, 3-Tert-Butyl-5-Chloro-2-Hydroxy-6-Methyl-N-(4-Nitro-2-Trifluoromethyl-Phenyl)-Benzamide, 3-Bromo-5-Tert-Butyl-N-(2-Chloro-4-Cyano-Phenyl)-6-Hydroxy-2-Methyl-Benzamide, 3-Bromo-5-Tert-Butyl-N-(2-Chloro-5-Trifluoromethyl-Phenyl)-6-Hydroxy-2-Methyl-Benzamide, 3-Bromo-5-Tert-Butyl-N-(2,4-Dichloro-Phenyl)-6-Hydroxy-2-Methyl-Benzamide, 3-Bromo-5-Tert-Butyl-N-(2,4-Dichloro-6-Nitro-Phenyl)-6-Hydroxy-2-Methyl-Benzamide, 3-Bromo-5-Tert-Butyl-N-(2,6-Dichloro-4-Nitro-Phenyl)-6-Hydroxy-2-Methyl-Benzamide, 3-Bromo-5-Tert-Butyl-N-[5-Chloro-4-[(4-Chloro-Phenyl)-Cyano-Methyl]-2-Methyl-Phenyl]-6-Hydroxy-2-Methyl-Benzamide, 3-Bromo-6-Hydroxy-5-Isopropyl-2-Methyl-N-(4-Nitro-2-Trifluoromethyl-Phenyl)-Benzamide, 3-Bromo-5-Tert-Butyl-6-Hydroxy-2-Methyl-N-(4-Nitro-2-Trifluoromethyl-Phenyl)-Benzamide, 3-Tert-Butyl-2-Hydroxy-6-Methyl-N-(4-Nitro-2-Trifluoromethyl-Phenyl)-Benzamide, 3-Bromo-N-(2-Bromo-3,5-Bis-Trifluoromethyl-Phenyl)-5-Tert-Butyl-6-Hydroxy-2-Methylbenzamide, N-(2,5-Bis-Trifluoromethyl-Phenyl)-3-Bromo-5-Tert-Butyl-6-Hydroxy-2-Methyl-Benzamide, 3-Bromo-5-Tert-Butyl-N-(2,4-Dichloro-6-Trifluoromethyl-Phenyl)-6-Hydroxy-2-Methylbenzamide, 3-Bromo-5-Tert-Butyl-6-Hydroxy-N-(4-Isopropyl-2-Trifluoromethyl-Phenyl)-2-Methyl-Benzamide, N-(3,5-Bis-Trifluoromethyl-Phenyl)-3-Fluoro-5-Trifluoromethyl-Benzamide, 3-Fluoro-N-(4-Nitro-3-Trifluoromethyl-Phenyl)-5-Trifluoromethyl-Benzamide, N-(3,5-Bis-Trifluoromethyl-Phenyl)-3-Fluoro-4-Trifluoromethyl-Benzamide, 4-Fluoro-N-(4-Nitro-3-Trifluoromethyl-Phenyl)-3-Trifluoromethyl-Benzamide, 3-(3,5-Di-Tert-Butyl-4-Hydroxy-Phenyl-2-(2,2-Dimethyl-Propionyl)-Acrylonitrile, 2-Acetyl-3-(3,5-Di-Tert-Butyl-4-Hydroxy-Phenyl)-Acrylic Acid Ethyl Ester, 2-(3,5-Dimethyl-4-Hydroxy-Benzylidene)-Malononitrile, 2-(3,5-Dimethyl-4-Hydroxy-Benzylidene)-Malononitrile, 2,6-Di-Tert-Butyl-4-Nitro-Phenol, 2-Tert-Butyl-4,6-Dinitro-Phenol, 3-(3,5-Di-Tert-Butyl-4-Hydroxy-Phenyl)-2-Pyridin-2-Yl-Acrylonitrile, 2-[1-(3,5-Di-Tert-Butyl-4-Hydroxy-Phenyl)-Ethylidene]-Malononitrile, 3-(3,5-Di-Tertbutyl-4-Hydroxybenzylidene)-2-(Diethylphosphonate)-Propenenitrile, 3-(3,5-Di-Tert-Butyl-4-Hydroxy-Phenyl)-2-(4-Nitro-Phenyl)-Acrylonitrile, 3-(3,5-Di-Tert-Butyl-4-Hydroxy-Phenyl)-2-Pyridin-4-Yl-Acrylonitrile, 2-(3,5-Bis-Trifluoromethyl-Phenyl)-3-(3,5-Di-Tert-Butyl-4-Hydroxy-Phenyl)-Acrylonitrile, 3-(3,5-Di-Tert-Butyl-4-Hydroxy-Phenyl)-2-(4-Trifluoromethoxy-Phenyl)-Acrylonitrile, 3-(3,5-Di-Tert-Butyl-4-Hydroxy-Phenyl)-2-(4-Trifluoromethyl-Phenyl)-Acrylonitrile, 2-Cyano-3-(3,5-Di-Tert-Butyl-4-Hydroxy-Phenyl)-But-2-Enoic Acid Ethyl Ester, N-(4-Chloro-Phenyl)-2-Cyano-3-(3,5-Di-Tert-Butyl-4-Hydroxy-Phenyl)-Acrylamide, (E)-3-(3,5-Di-Tert-Butyl-4-Hydroxy-Phenyl)-2-Methanesulfonyl-Acrylonitrile, (E)-2-(4-Chloro-Benzene sulfonyl)-3-(3,5-Di-Tert-Butyl-4-Hydroxy-Phenyl)-Acrylonitrile, (E)-3-(3,5-Di-Tert-Butyl-4-HydroxyPhenyl)-2-(4-Fluoro-Benzenesulfonyl)-Acrylonitrile, (E)-2-Benzenesulfonyl-3-(3,5-Di-Tert-Butyl-4-Hydroxy-Phenyl)-Acrylonitrile, (E)-3-(3,5-Di-Tert-Butyl-4-Hydroxy-Phenyl)-2-(Propane-2-Sulfonyl)-Acrylonitrile, 3-(3,5-Di-Tert-Butyl-4-Hydroxy-Phenyl)-2-(2,5-Dichloro-Benzenesulfonyl)-Acrylonitrile, 3-(3,5-Di-Tert-Butyl-4-Hydroxy-Phenyl)-2-(2,4-Dichloro-Benzenesulfonyl)-Acrylonitrile, 3-(3,5-Di-Tert-Butyl-4-Hydroxy-Phenyl)-2-(Hexane-1-Sulfonyl)-Acrylonitrile, 2-(4-Bromo-Benzenesulfonyl)-3-(3,5-Di-Tert-Butyl-4-Hydroxy-Phenyl)-Acrylonitrile, 2-(3-Bromo-Benzenesulfonyl)-3-(3,5-Di-Tert-Butyl-4-Hydroxy-Phenyl)-Acrylonitrile, 5-Bromo-3-Tert-Butyl-N-(2-Chloro-4-Cyanophenyl)-2-Hydroxybenzamide, 5-Bromo-3-Tert-Butyl-N-(4-Cyanophenyl)-2-Hydroxybenzamide, 3-Bromo-5-Tert-Butyl-6-Hydroxy-2-Methyl-N-(2-Trifluoromethylphenyl)Benzamide, 2-(2-Bromo-1H-Indol-3-Ylmethylene)Malononitrile, 2-(7-Bromo-2-Methyl-1H-Indol-3-Ylmethylene)Malononitrile, 2-(5-Bromo-2-Methyl-1H-Indo1-3-Ylmethylene) Malononitrile, pharmaceutically acceptable salts and its derivatives, and mixtures thereof.

14. The method for the measurement, diagnosis, treatment, and management of metabolic syndrome comprising the administration of a drug product to the patient (100) in need thereof to prevent or reduce the risk of metabolic syndrome and related disorders, by using the electronic devices according to claim 1, wherein the pharmaceutical composition with a drug product (130) comprises drug products for the prevention, reduction, or control of hypertension, selected from the group consisting of: alprenolol, atenolol, timolol, pindolol, propranolol and metoprolol, ACE (angiotensin converting enzyme) inhibitors, calcium channel blockers, and [alpha]-blockers, pharmaceutically acceptable salts and its derivatives, and mixtures thereof.

15. The method for measurement, diagnosis, treatment, and management of metabolic syndrome comprising the administration of a drug product to the patient (100) in need thereof to prevent or reduce the risk of metabolic syndrome and related disorders by using electronic devices according to claim 1, wherein the pharmaceutical composition with a drug (130) comprises drug products for the prevention, reduction, or control of hyperglycemia (type I diabetes or type II diabetes), selected from the group consisting of: chemical uncouplers of oxidative phosphorylation or derivatives thereof, agents acting on the ATP-dependent potassium channel of pancreatic [beta] cells, biguanides, imidazolines, [alpha]-glucosidase inhibitors, meglitinides, oxadiazolidinediones, thiazolidinediones, insulin sensitizers, sulfonylureas, potassium channel blockers, glucagon antagonists, DPP-IV (dipeptidyl peptidase-IV) inhibitors, PTPase (protein tyrosine phosphatase) inhibitors, glucokinase activators, inhibitors of liver enzymes involved in the stimulation of gluconeogenesis and/or glycogenolysis, glucose uptake modulators, GSK-3 (glycogen synthase kinase-3) inhibitors, compounds that modify lipid metabolism, compounds that reduce food intake and PPAR agonists; tyrphostin AG17, tyrphostin A9, insulin and insulin analogs, tolbutamide, chlorpropamide, tolazamide, glibenclamide, glipizide, glimepiride, glicazide o gliburide, metformin, repaglinide or senaglinide/nateglinide, troglitazone, pioglitazone, rosiglitazone, isaglitazone, darglitazone, englitazone, GI 262570, YM-440, MCC-555, JTT-501, AR-H039242, KRP-297, GW-409544, CRE-16336, AR-H, voglibose, emiglitate, miglitol o acarbose, tolbutamide, glibenclamide, glipizide, glicazide, BTS-67582 o repaglinide, nateglinide, colestiramine, colestipol, clofibrate, gemfibrozil, lovastatine, pravastatine, simvastatine, pharmaceutically acceptable salts and its derivatives, and mixtures thereof.

16. A method for measurement, diagnosis, treatment, and management of overweight and obesity comprising administration of a drug product to a patient (100) in need thereof to prevent or reduce a risk of overweight and obesity by using electronic devices, the method comprising:
(a) collecting (101) and storing patient identity information (102a) and/or information of body metrics, clinical parameters, and/or anthropometric indicators (102c) in a database (103) for incorporation of a medical record (102b);
(b) linking (111) the patient (100) with a health care professional (110) specialist in overweight and obesity, for the assessment of the patient's health status;
(c) indicating to the patient the patient's health status (111b) and record in the medical record (102b);
(d) instructing the patient (111c) indications for the treatment of overweight and obesity that the patient suffers or for the prevention of overweight and obesity;
(e) through a pharmacy (120), providing (121a) to the patient (100) in need thereof, a pharmaceutical composition with a useful drug product (130) for the prevention, reduction, or control of overweight and obesity;
(f) through the pharmacy (120), providing (121b) to the patient (100) in need thereof, supplies (140a) or measuring devices (140) for the prevention, reduction, or control of overweight and obesity;
(g) connecting (151) by using an interactive user interface (150) one or more devices for measuring clinical parameters and/or anthropometric indicators (140b);
(h) measuring (141), recording (142), and storing (143) in the database (103) the collected clinical parameters and/or anthropometric indicators (102c);
(i) monitoring (112) the data of the clinical parameters and/or anthropometric indicators (102c) of the patient (100) by the health care professional (110) specialist in overweight and obesity for the assessment of the patient's health status;
(j) adjusting or maintaining the indications (113) for the treatment of overweight and obesity suffered by the patient or for the prevention of overweight and obesity; and
(k) optionally, assisting (114) to the patient (100) by the health care professional (110) at established periods of time or at the request of the patient or the health care professional.

17. A method for measurement, diagnosis, treatment, and management of overweight and obesity comprising administration of a drug product to a patient in need thereof to prevent or reduce a risk of overweight and obesity, by using electronic devices, the method comprising:
(a) providing (121a) to the patient (100) a pharmaceutical composition with a useful drug product (130) for the prevention, reduction, or control of overweight and obesity, from patient identity information (102a) and body metrics information (102c) and/or a medical record (102b) of the patient previously stored in a database (103); and
(b) monitoring (112) data of clinical parameters and/or anthropometric indicators of the patient by a health care professional specialist (110) in overweight and obesity, for the assessment of the patient's health status by using an interactive user interface (150) connected with one or more devices for measuring clinical parameters and/or anthropometric indicators, wherein the interactive user interface (150) records (142) and stores (143) clinical parameters and/or anthropometric indicators.

18. A method for measurement, diagnosis, treatment, and management of overweight and obesity comprising administration of a drug product to a patient undergoing treatment prevention, reduction, or control of overweight and obesity, by using electronic devices, the method comprising:
(a) collecting (101) and storing patient identity information (102a) and/or information of body metrics, clinical parameters, and/or anthropometric indicators (102c) in a database (103) for the incorporation of a medical record (102b);
(b) through a pharmacy (120), providing (121a) to the patient (100) in need thereof, a pharmaceutical composition with a useful drug product (130) for the prevention, reduction, or control of overweight and obesity from the patient identity information (102a) and the patient clinical parameters and/or anthropometric indicators information (102c) and/or the medical record (102b) of the patient previously stored in a database;
(c) measuring (141), recording (142), and storing (143) in the database (103) the collected clinical parameters and/or anthropometric indicators (102c) of the patient during treatment;
(d) monitoring (112) the data of the clinical parameters and/or anthropometric indicators (102c) of the patient (100) by a health care professional (110) specialist in overweight and obesity for the assessment of their health status; and
(e) adjusting or maintaining indications (113) for the treatment of overweight and obesity suffered by the patient or for the prevention of overweight and obesity.

19. A method for measurement, diagnosis, treatment, and management of overweight and obesity comprising administration of a drug product to a patient in need thereof to prevent or reduce a risk of overweight and obesity by using electronic devices, the method comprising:
(a) linking (111) the patient (100) with a health care professional (110) specialist in overweight and obesity, for the assessment of the patient's health status;
(b) indicating to the patient the patient's health status (111b) and recording in the medical record (102b);
(c) instructing the patient (111c) indications for the treatment of overweight and obesity that the patient suffers or for the prevention of overweight and obesity;
(d) through a pharmacy (120), providing (121a) to the patient (100) in need thereof, a pharmaceutical composition with a useful drug product (130) for the prevention, reduction, or control of overweight and obesity;
(e) recording (142) and storing (143) in the database (103) body metrics, clinical parameters and/or anthropometric indicators collected (102c) from the patient during treatment;
(f) monitoring (112) the data of the clinical parameters and/or anthropometric indicators of the patient (102c) (100) by the health care professional (110) specialist in overweight and obesity for assessment of the patient's health status and
(g) adjusting or maintaining indications (113) for the treatment of overweight and obesity suffered by the patient or for the prevention of overweight and obesity.

20. A method for measurement, diagnosis, treatment, and management of overweight and obesity comprising the administration of a drug product to a patient in need thereof to prevent or reduce a risk of overweight and obesity, by using electronic devices, the method comprising:
(a) through a pharmacy (120), providing (121a) to the patient (100) in need thereof, a pharmaceutical composition with a useful drug product (130) for the prevention, reduction, or control of overweight and obesity;
(b) through the pharmacy (120), providing (121b) to the patient (100) in need thereof, supplies (140a) or supportive devices (140b) for the prevention, reduction, or control of overweight and obesity;
(c) connecting (151) by using an interactive user interface (150) one or more devices for measuring clinical parameters and/or anthropometric indicators (140b);
(d) measuring (141), recording (142), and storing (143) in the database (103) the collected clinical parameters and/or anthropometric indicators (102c);
(e) monitoring (112) the data (100) clinical parameters and/or anthropometric indicators of the patient (102c) by the health care professional (110) specialist in overweight and obesity for assessment of the patient's health status; and
(f) adjusting or maintaining indications (113) for the treatment of overweight and obesity suffered by the patient or for the prevention of overweight and obesity.

21. A method for measurement, diagnosis, treatment, and management of overweight and obesity comprising administration of a drug to a patient in need thereof, to prevent or reduce a risk of overweight and obesity, by using electronic devices, the method comprising:
(a) through a pharmacy (120), providing (121a) to the patient (100) in need thereof, a pharmaceutical composition with a useful drug product (130) for the prevention, reduction, or control of overweight and obesity disorders;
(b) connecting (151) by using an interactive user interface (150) one or more devices for measuring clinical parameters and/or anthropometric indicators (140b);
(c) measuring (141), recording (142), and storing (143) in the database (103) the collected clinical parameters and/or anthropometric indicators (102c);
(d) monitoring (112) the data (100) clinical parameters and/or anthropometric indicators of the patient (102c) by the health care professional (110) specialist in overweight and obesity for assessment of the patient's health status and
(e) adjusting or maintaining indications (113) for the treatment of overweight and obesity suffered by the patient or for the prevention of overweight and obesity.

22. A system for monitoring at least one clinical parameter and/or anthropometric indicator to a patient (100) in need thereof to prevent or reduce risk of overweight and obesity, by using an electronic device associated with devices for measuring clinical parameters and/or anthropometric indicators, the system comprising:
(a) a patient monitoring module installed in the electronic device (160a) of the patient;
(b) at least one device for measuring clinical parameters and/or anthropometric indicators (140) with connectivity to the patient electronic device (160a);
(c) a means of liaison (180) between the electronic device (160a) of the patient and the device for measuring clinical parameters and/or anthropometric indicators (140),
wherein the patient monitoring module is configured to:
(i) collect (101) patient identity information (102a) and/or body metrics information (102c);
(ii) measure (141), record (142), and store (143) in the database (103) the collected clinical parameters and/or anthropometric indicators (102c) of the patient;
(iii) store the information in a database (103) for incorporation into a medical record (102b);
(iv) link (111) the patient (100) with a health care professional (110) specialist in overweight and obesity, for the assessment of the patient's health status, indicating to the patient the patient's health status (111b), recording in the medical record (102b), and instruct the patient (111c) the indications for the treatment of overweight and obesity from which the patient suffers or for the prevention of overweight and obesity;
(v) through a pharmacy (120), provide (121a) to the patient (100) in need thereof, a pharmaceutical composition with a useful drug product (130) for the prevention, reduction, or control of overweight and obesity, and supplies (140a) or support devices (140b);
(vi) to establish a connection (151) with at least one device for measuring clinical parameters and/or anthropometric indicators (140);
(vii) measure (141), record (142), and store (143) in the database (103) the clinical parameters and/or anthropometric indicators (102c) obtained from at least one device for measuring clinical parameters and/or anthropometric indicators (140);
(viii) allow access (170) to the health care professional specialist in overweight and obesity (110) to the patient medical record for the assessment of the patient's health status and the adjustment or maintenance (113) of indications for treatment; and
(ix) optionally, establishing communication (114) between the patient (100) and the health care professional (110) at established periods of time or upon request of the patient or the health care professional.

23. A system for monitoring at least one clinical parameter and/or anthropometric indicator to a patient (100) in need thereof, to prevent or reduce risk of overweight and obesity, by using electronic devices, the system comprising:
(a) a device and interface for entering patient identity information and/or body metrics information into a database (160a);
(b) a device and interface to collect and store the patient identity information and/or the body metrics information in a database for incorporation of a medical record (160b);
(c) means of access for liaison between the patient and a health care professional specialist in overweight and obesity for assessment of the patient's health status (170);
(d) means to indicate to the patient the patient's health status and record in the medical record (170a);
(e) means to instruct the patient on indications for the treatment of overweight and obesity that the patient suffers or for the prevention of overweight and obesity (170b);
(f) means to provide to the patient in need thereof (120), a pharmaceutical composition containing a useful drug product for the prevention, reduction, or control of overweight and obesity (121a);
(g) means to provide to the patient in need thereof, supplies or supportive devices for the prevention, reduction, or control of overweight and obesity (121b);
(h) means of connection by using an interactive user interface for one or more devices for measuring clinical parameters and/or anthropometric indicators (152);
(i) a database for recording and storing the collected clinical parameters and/or anthropometric indicators (103);
(j) means of monitoring the data of the clinical parameters and/or anthropometric indicators of the patient by a health care professional specialist in overweight and obesity for the evaluation of their health status (112);
(k) means of access for the health care professional to adjust or maintain the indications for the treatment of overweight and obesity suffered by the patient or for the prevention of overweight and obesity (113); and
(l) optionally, means of access to establish communication between the patient and the health care professional at set periods of time or at the request of the patient or the health care professional (114).

24. A method for measurement, diagnosis, treatment, and management of overweight and obesity comprising the administration of a drug product to a patient in need thereof to prevent or reduce the risk of overweight and obesity by using electronic devices, the method comprising:
(a) providing a monitoring system (200) for treatment or prevention to the patient and a health care professional comprising at least: a device and interface to enter patient identity information and/or body metrics information into a database (160a); a device and interface to collect and store patient identity information and/or body metrics information in a database for incorporation of a medical record (160b); means of access for liaison between the patient and the health care professional specialist in metabolic syndrome and related disorders for assessment of the patient's health status (170); means to indicate to the patient the patient's health status and record in the medical record (170a); means to instruct the patient on indications for the treatment of overweight and obesity that the patient suffers or for the prevention of overweight and obesity (170b); means to provide to the patient in need thereof (120), a pharmaceutical composition with a useful drug product for the prevention, reduction, or control of overweight and obesity (121a); means to provide to the patient in need thereof supplies or supportive devices for the prevention, reduction, or control of overweight and obesity (121b); means of connection by using an interactive user interface for one or more devices for measuring clinical parameters and/or anthropometric indicators (152); a database for recording and storing the collected clinical parameters and/or anthropometric indicators (103); means of monitoring the data of the clinical parameters and/or anthropometric indicators of the patient by the health care professional specialist in overweight and obesity for the evaluation of the patient's health status (112); means of access for the health care professional to adjust or maintain the indications for the treatment of overweight and obesity suffered by the patient or for the prevention of overweight and obesity (113); and optionally, means of access to establish communication between the patient and the health care professional at established time periods or at the request of the patient or the health care professional (114);
(b) providing the patient (100) with a pharmaceutical composition with a useful drug product (130) for the prevention, reduction, or control of overweight and obesity from the patient identity information and body metrics information or the medical record of the patient previously stored in a database; and
(c) monitoring the data of clinical parameters and/or anthropometric indicators of the patient by the healthcare professional (110) specialist in metabolic syndrome and related disorders for the assessment of their health status by using an interactive user interface connected to one or more devices for measuring clinical parameters and/or anthropometric indicators.

25. The method for the measurement, diagnosis, treatment, and management of overweight and obesity comprising the administration of the drug product to the patient (100) in need thereof to prevent or reduce the risk of overweight and obesity by using electronic devices, according to any one of claims 16 to 21, wherein the body metrics, clinical parameters, and/or anthropometric indicators are selected from the group consisting of: weight, body mass index, % fat, height, muscle, water, visceral fat, bone, metabolism, protein, degree of obesity, body age, fat-free weight, oxygenation, body oxygenation quantified in urine, vitamins and minerals, blood glucose, blood pressure, LDL cholesterol, HDL cholesterol, triglycerides, urine glucose, step counter, heart rate, fasting glucose, post-load glucose, total cholesterol, total bilirubin, alanine transaminase, aspartate aminotransferase, body temperature, and any combination thereof.

26. The method for the measurement, diagnosis, treatment, and management of overweight and obesity comprising the administration of the drug product to the patient (100) in need thereof to prevent or reduce the risk of overweight and obesity by using electronic devices, according to any one of claims 16 to 21, wherein the measuring devices (140) are selected from the group consisting of: scale, ORP, Vitamin stick, glucometer, oximeter, smart watch, smart bracelet, smart clothing, patches, tattoos, implants, hearing aids, helmets, electrodes, chest band, pedometer, medical equipment for quantification of biochemical parameters, and any combination thereof.

27. The method for the measurement, diagnosis, treatment, and management of overweight and obesity comprising the administration of the drug to the patient (100) in need thereof to prevent or reduce the risk of overweight and obesity by using electronic devices, according to any one of claims 16 to 21, wherein the pharmaceutical composition with the drug product (130) useful for the prevention, reduction, or control of overweight and obesity is selected from the group consisting of: chemical uncouplers of oxidative phosphorylation, tyrphostin AG17, orlistat, L-carnitine, benzphetamine, phendimetrazine, diethylpropion, liraglutide, naltrexone, phentermine, topiramate, glibenclamide, metformin, acarbose, canagliflozin, dapagliflozin, saxagliptin, dulaglutide, empagliflozin, exenatide, lixisenatide, semaglutide, CBD, sibutramin, hydroxycitric acid, coenzyme q10, chromium picolinate, gamma-linolenic acid, resveratrol, omega-3 fatty acid, antioxidants, vitamins, vitamin B, vitamin C, adrenal glandular extract, pantothenic acid, Curcuma longa, Bayberry bark, Myrica cerifera, Oryza sativa L. indica, Camellia sinensis, Theobroma cacao, Emblica officinalis, Ilex paraguariensis, Cascara sagrada bark, Rhamnus purshiana, Acacia nilotica, persimmon, Blueberry leaf, Grape seed, Cordia salicifolia, Hypericum perforatum, Pueruria thomsonn, Capparis spinosa, Gymnema sylvestre, Elderberry, Sambicus, Citrus aurantium, Coffea canefora, Sygium cumini, Rosmarinus officinalis, Zingiber officinalis, Cassia nomame, Cissus quadrangularis, Phaseolus vulgaris, Licorice Root, Fenugreek, Yohimbe, White willow, Cordyceps sinensis, Ashwaganda, Astragalus, Gingseng, Schisandra, Siberian gingseng, Asian gingseng, Commiphora mukul, Momordica charantia, Garcinia cambogia, Althaea officinalis, Bougainvillea spectabilis, Medicago sativa, Valeriana officinalis, Damiana, Sambucus nigra, Capsicum genus, Allium genus, calcium myristate, magnesium myristate, tetradecylthioacetic acid (TTA), thyroid hormones or their precursors, enhancers of thyroid function, Kreb's cycle metabolites, endogenous uncoupling protein(s) or its (their) precursor(s), agonist(s), or enhancers, leucine, valine, isoleucine, glutamine, proline, tyrosine, conjugated linoleic acid, adrenaline secretion enhancers, fatty acids or esters, beta agonists, glucagon, arbutamine, vasopressin, ubiquinone, coenzyme 01, coenzyme Q2, melatonin, fatty acid esters of estrogens, insulin or insulin analogues and orally active hypoglycemic agents;
acetohexamide, carbutamide, chlorpropamide, glibenclamide, glibornuride, gliclazide, glimepiride, glipizide, tolazamide, and tolbutamide. 2R,3R,4R,5S)-1-(2-hydroxyethyl)-2-(hydroxymethyl)piperidine-3,4,5-triol or Glyset) (1S,2S,3R,4S,5S)-5-(1,3-dihydroxypropan-2-ylamino)-1-(hydroxymethyl)-cyclohexane-1,2,3,4-tetraol), (RS)-5-[4-(2-[methyl(pyridin-2-yl)amino]ethoxy)benzyl]tiazo-lidine-2,4-di-one, (RS)-5-(4-[2-(5-ethylpyridin-2-yl)ethoxy]benzyl)thiazolidine-2,4-dione, 5-[(4-[2-([6-(4-Methoxyphenoxyl)pyrimidin-4-yl]-methylamino)ethoxy]phenyl-)methyl]-1,3-thiazolidine-2,4-dione, (RS)-5-(4-[(6-hydroxy-2,5,7,8-tetramethylchroman-2-yl)methoxy]benzyl)thia-zolidine-2,4-dione. (S)-(+)-2-ethoxy-4-[2-(3-methyl-1-[2-(piperidin-1-yl)phenyl]butylamino)-2--oxoethyl]benzoic acid, 3-phenyl-2-[(4-propan-2-ylcyclohexanecarbonyl)amino]propanoic acid, (2S)-2-benzyl-4-[(3aR,7aS)-octahydro-2H-isoindo1-2-yl]-4-oxobutanoic acid, L-ornithine, L-tyrosine, L-tryptophan, L-phenylalanine, conjugated linoleic acid, gamma-linolenic acid, chromium picolinate, glucose tolerance factor, vanadyl sulfate, Gymnema sylvestre, bromelain, pancreatin, papain, coenzyme Q10, curcumin, barberry, bearberry, Silymarin, Teucrium polium, choline, inositol, human growth hormone, DHEA (dehydroepiandrosterone), caffeine, xanthenes (e.g., fucoxanthin), kola nut, psyllium, yerba mate, guarana, ginseng, medium chain triglycerides, hydroxycitric acid (HCA), kelp, lecithin, dihydroxyacetone, pyruvate, creatine, iodine, niacin, bladderwrack, white bean extract, glucomannan, chitosan, resveratrol, resveratrol derivatives, vitamin D, hCG, capsaicin, chia, apple cider vinegar, coconut oil, bitter orange, B vitamin, fluoxetine, chitosan, chromium picolinate, conjugated linoleic acid (CLA), glucomannan, green tea extract, green coffee extract, guar gum, hoodia, 7-keto-DHEA, DHEA, ephedra, bitter orange, turmetic, ginger, root powder, apple cider vinegar, calcium, hesperidin, apantipan gynostemma, extract flaxseed oil, orange peel powder, thiamin, ashwagandha extract, coffee extract, L-theanine, choline bitartrate, red mango extract, white kidney bean extract, c. cinephora robusta extract, acetyl—L carnitine, garcinia, cumbusia extract, coconut oil, forskolin extract, black pepper extract seroxat, citalopram, bupropion, topiramate, ecopipam, naltrexone, peptide YY3-36, leptin, examphetamine, amphetamine, phentermine, mazindol phendimetrazine, diethylpropion, fenfluramine, dexfenfluramine, pharmaceutically acceptable salts and its derivatives, and mixtures thereof.

28. The method for the measurement, diagnosis, treatment, and management of overweight and obesity comprising the administration of the drug product to the patient (100) in need thereof to prevent or reduce the risk of overweight and obesity using electronic devices according to claim 27, wherein the oxidative phosphorylation chemical uncouplers is selected from the group consisting of: 4,4-Bis-(4-Hydroxy-3-Nitrophenyl)-Valeric Acid, 4-Methoxy-2-Nitrophenol, 4-Hydroxy-3-Nitroacetophenone, 7-Hydroxy-4-Methyl-8-Nitro-Chromen-2-One, 3-Tert-Butyl-5-Chloro-N-(2-Chloro-4-Nitrophenyl)-2-Hydroxy-6-Methyl-Benzamide, N-1-[4-Cyano-3-(Trifluoromethyl)Phenyl]-3,5-Di(Trifluoromethyl)Benzamide, N-(4-Cyanophenyl)Benzamide, 2′-Chloro-1-Hydroxy-4′-Nitro-2-Naphthanilide, N-(2-Chloro-4-Bromophenyl)-5-Bromosalicylanilide, N-(2-Chloro-4-Nitrophenyl)-3-Tert-Butyl-6-Methylsalicylanilide, (3,5-Di-Tert-Butyl-4-Hydroxybenzyl) Triphenylphosphonium Bromide, 3,5-Di-Tert-Butyl-4-Hydroxybenzyl)Tricyclohexylpho sphonium Bromide, (3,5-Di-Tert-Butyl-4-Hydroxybenzyl)Tributylphosphonium Bromide, (3,5-Di-Tert-Buty4-Hydroxybenzyl)Trioctylphosphonium Bromide, 2-Cyano-3-(3,5-Di-Tert-Butyl-4-Hydroxyphenyl)-Acrylic Acid Ethyl Ester, 2-(3,5-Di-Tert-Butyl-4-Hydroxy-Benzylidene)-Malonic Acid Diethyl Ester, 2-Amino-S-[(3,5-Di-Tert-Butyl-4-Hydroxybenzylidone)-Amino]-But-2-Enedinitrile, 2-(3,5-Di-Tert-Butyl-4-Hydroxy-Benzylidene)-Indan-1,3-Dione, 2-[[2-(4-Chlorophenyl)-1H-Indol-3-Yl]Methylene]Malononitrile, 2-(4-Chlorophenyl)-Indole, N-(2,4,5-Trichlorophenyl)Salicylanilide, 2,3-Dimethyl-5-Cyano-7-Ethylindole, 4-Bromo-2-(4-Chlorophenyl)-5-Trifluoromethyl-1H-Pyrrole-3-Carbonitrile, N-(3-Cyano-4-Phenylsulfanyl-Phenyl)-3-Trifluoromethyl-Benzamide, 2,4-Dinitrophenol, Carbonylcyanide p-Trifluoromethoxy-Phenylhydrazone, 2-(5,7-Dimethyl-1H-Indol-3-Ylmethylene)-Malononitrile, 2-(5-Bromo-1H-Indol-3-Ylmethylene)-Malononitrile, 2-((5-Chloro-1H-Indol-3-Yl)Methylene)Malononitrile, 2-((5-Methyl-1H-Indol-3-Yl)Methylene)Malononitrile, 2-((5-Methyl-1H-Indol-3-Yl)Methylene)Malononitrile, 2-(2-Phenyl-3-Indolylmethylene)-Malononitrile, 2-(2-Chloro-1H-Indol-3-Ylmethylene)-Malononitrile, 2-(5-Nitro-1H-Indol-3-Ylmethylene)-Malononitrile, 2-(2-Methyl-5-Nitro-1H-Indol-3-Ylmethylene)-Malononitrile, 3-Bromo-5-Tert-Butyl-N-(2-Chloro-4-Nitro-Phenyl)-6-Hydroxy-2-Methyl-Benzamide, N-(2-Chloro-4-Nitro-Phenyl)-2-Hydroxy-3-Isopropyl-Benzamide, N-(2-Chloro-4-Nitro-Phenyl)-2-Hydroxy-3-Isopropyl-6-Methyl-Benzamide, 3,5-Di-Tert-Butyl-N-(2-Chloro-4-Nitro-Phenyl)-2-Hydroxy-Benzamide, 3-Bromo-N-(2-Chloro-4-Nitro-Phenyl)-6-Hydroxy-5-Isopropyl-2-Methyl-Benzamide, 3-Tert-Butyl-5-Chloro-N-(4-Chloro-3-Trifluoromethyl-Phenyl)-2-Hydroxy-6-Methyl-Benzamide, 3-Tert-Butyl-5-Chloro-N-(4-Cyano-3-Trifluoromethyl-Phenyl)-2-Hydroxy-6-Methyl-Benzamide, 2-Hydroxy-Biphenyl-3-Carboxylic Acid (2-Chloro-4-Nitro-Phenyl)-Amide, 3-Tert-Butyl-N-(2-Chloro-4-Nitro-Phenyl)-2-Hydroxy-5-Methyl-Benzamide, N-(2-Chloro-4-Nitro-Phenyl)-2-Hydroxy-6-Isopropyl-3-Methyl-Benzamide, N-(3,5-Bis-Trifluoromethyl-Phenyl)-3-Tert-Butyl-5-Chloro-2-Hydroxy-6-Methyl-Benzamide, 3-Tert-Butyl-5-Chloro-N-(2-Fluoro-5-Trifluoromethyl-Phenyl)-2-Hydroxy-6-Methyl-Benzamide, 3-Tert-Butyl-5-Chloro-2-Hydroxy-6-Methyl-N-(4-Nitro-3-Trifluoromethyl-Phenyl)-Benzamide, 3-Tert-Butyl-5-Chloro-2-Hydroxy-6-Methyl-N-(4-Nitro-2-Trifluoromethyl-Phenyl)-Benzamide, 3-Bromo-5-Tert-Butyl-N-(2-Chloro-4-Cyano-Phenyl)-6-Hydroxy-2-Methyl-Benzamide, 3-Bromo-5-Tert-Butyl-N-(2-Chloro-5-Trifluoromethyl-Phenyl)-6-Hydroxy-2-Methyl-Benzamide, 3-Bromo-5-Tert-Butyl-N-(2,4-Dichloro-Phenyl)-6-Hydroxy-2-Methyl-Benzamide, 3-Bromo-5-Tert-Butyl-N-(2,4-Dichloro-6-Nitro-Phenyl)-6-Hydroxy-2-Methyl-Benzamide, 3-Bromo-5-Tert-Butyl-N-(2,6-Dichloro-4 -Nitro-Phenyl)-6-Hydroxy-2-Methyl-Benzamide, 3-Bromo-5-Tert-Butyl-N-[5-Chloro-4-{(4-Chloro-Phenyl)-Cyano-Methyl]-2-Methyl-Phenyl}-6-Hydroxy-2-Methyl-Benzamide, 3-Bromo-6-Hydroxy-5-Isopropyl-2-Methyl-N-(4-Nitro-2-Trifluoromethyl-Phenyl)-Benzamide, 3-Bromo-5-Tert-Butyl-6-Hydroxy-2-Methyl-N-(4-Nitro-2-Trifluoromethyl-Phenyl)-Benzamide, 3-Tert-Butyl-2-Hydroxy-6-Methyl-N-(4-Nitro-2-Trifluoromethyl-Phenyl)-Benzamide, 3-Bromo-N-(2-Bromo-3,5-Bis-Trifluoromethyl-Phenyl)-5-Tert-Butyl-6-Hydroxy-2-Methylbenzamide, N-(2,5-Bis-Trifluoromethyl-Phenyl)-3-Bromo-5-Tert-Butyl-6-Hydroxy-2-Methyl-Benzamide, 3-Bromo-5-Tert-Butyl-N-(2,4-Dichloro-6-Trifluoromethyl-Phenyl)-6-Hydroxy-2-Methylbenzamide, 3-Bromo-5-Tert-Butyl-6-Hydroxy-N-(4-Isopropyl-2-Trifluoromethyl-Phenyl)-2-Methyl-Benzamide, N-(3,5-Bis-Trifluoromethyl-Phenyl)-3-Fluoro-5-Trifluoromethyl-Benzamide, 3-Fluoro-N-(4-Nitro-3-Trifluoromethyl-Phenyl)-5-Trifluoromethyl-Benzamide, N-(3,5-Bis-Trifluoromethyl-Phenyl)-3-Fluoro-4-Trifluoromethyl-Benzamide, 4-Fluoro-N-(4-Nitro-3-Trifluoromethyl-Phenyl)-3-Trifluoromethyl-Benzamide, 3-(3,5-Di-Tert-Butyl-4-Hydroxy-Phenyl-2-(2,2-Dimethyl-Propionyl)-Acrylonitrile, 2-Acetyl-3-(3,5-Di-Tert-Butyl-4-Hydroxy-Phenyl)-Acrylic Acid Ethyl Ester, 2-(3,5-Dimethyl-4-Hydroxy-Benzylidene)-Malononitrile, 2-(3,5-Dimethyl-4-Hydroxy-Benzylidene)-Malononitrile, 2,6-Di-Tert-Butyl-4-Nitro-Phenol, 2-Tert-Butyl-4,6-Dinitro-Phenol, 3-(3,5-Di-Tert-Butyl-4-Hydroxy-Phenyl)-2-Pyridin-2-Yl-Acrylonitrile, 2-[1-(3,5-Di-Tert-Butyl-4-Hydroxy-Phenyl)-Ethylidene]-Malononitrile, 3-(3,5-Di-Tertbutyl-4-Hydroxybenzylidene)-2-(Diethylphosphonate)-Propenenitrile, 3-(3,5-Di-Tert-Butyl-4-Hydroxy-Phenyl)-2-(4-Nitro-Phenyl)-Acrylonitrile, 3-(3,5-Di-Tert-Butyl-4-Hydroxy-Phenyl)-2-Pyridin-4-Yl-Acrylonitrile, 2-(3,5-Bis-Trifluoromethyl-Phenyl)-3-(3,5-Di-Tert-Butyl-4-Hydroxy-Phenyl)-Acrylonitrile, 3-(3,5-Di-Tert-Butyl-4-Hydroxy-Phenyl)-2-(4-Trifluoromethoxy-Phenyl)-Acrylonitrile, 3-(3,5-Di-Tert-Butyl-4-Hydroxy-Phenyl)-2-(4-Trifluoromethyl-Phenyl)-Acrylonitrile, 2-Cyano-3-(3,5-Di-Tert-Butyl-4-Hydroxy-Phenyl)-But-2-Enoic Acid Ethyl Ester, N-(4-Chloro-Phenyl)-2-Cyano-3-(3,5-Di-Tert-Butyl-4-Hydroxy-Phenyl)-Acrylamide, (E)-3-(3,5-Di-Tert-Butyl-4-Hydroxy-Phenyl)-2-Methanesulfonyl-Acrylonitrile, (E)-2-(4-Chloro-Benzenesulfonyl)-3-(3,5-Di-Tert-Butyl-4-Hydroxy-Phenyl)-Acrylonitrile, (E)-3-(3,5-Di-Tert-Butyl-4-HydroxyPhenyl)-2-(4-Fluoro-Benzenesulfonyl)-Acrylonitrile, (E)-2-Benzene sulfonyl-3-(3,5-Di-Tert-Butyl-4-Hydroxy-Phenyl)-Acrylonitrile, (E)-3-(3,5-Di-Tert-Butyl-4-Hydroxy-Phenyl)-2-(Propane-2-Sulfonyl)-Acrylonitrile, 3-(3,5-Di-Tert-Butyl-4-Hydroxy-Phenyl)-2-(2,5-Dichloro-Benzenesulfonyl)-Acrylonitrile, 3-(3,5-Di-Tert-Butyl-4-Hydroxy-Phenyl)-2-(2,4-Dichloro-Benzenesulfonyl)-Acrylonitrile, 3-(3,5-Di-Tert-Butyl-4-Hydroxy-Phenyl)-2-(Hexane-1-Sulfonyl)-Acrylonitrile, 2-(4-Bromo-Benzenesulfonyl)-3-(3,5-Di-Tert-Butyl-4-Hydroxy-Phenyl)-Acrylonitrile, 2-(3 -Bromo-Benzenesulfonyl)-3-(3,5-Di-Tert-Butyl-4-Hydroxy-Phenyl)-Acrylonitrile, 5-Bromo-3-Tert-Butyl-N-(2-Chloro-4-Cyanophenyl)-2-Hydroxybenzamide, 5-Bromo-3-Tert-Butyl-N-(4-Cyanophenyl)-2-Hydroxybenzamide, 3-Bromo-5-Tert-Butyl-6-Hydroxy-2-Methyl-N-(2-Trifluoromethylphenyl)Benzamide, 2-(2-Bromo-1H-Indol-3-Ylmethylene)Malononitrile, 2-(7-Bromo-2-Methyl-1H-Indol-3-Ylmethylene)Malononitrile, 2-(5-Bromo-2-Methyl-1H-Indol-3-Ylmethylene) Malononitrile, pharmaceutically acceptable salts and its derivatives, and mixtures thereof.

29. The method for the measurement, diagnosis, treatment, and management of overweight and obesity comprising the administration of the drug product to the patient (100) in need thereof to prevent or reduce the risk of overweight and obesity by using electronic devices, according to any one of claims 16 to 21, wherein the pharmaceutical composition with the drug product (130) comprises drug products for the prevention, reduction, or control of hypertension, selected from the group consisting of: alprenolol, atenolol, timolol, pindolol, propranolol and metoprolol, ACE (angiotensin converting enzyme) inhibitors, calcium channel blockers, and [alpha]-blockers, pharmaceutically acceptable salts and its derivatives, and mixtures thereof.

30. The method for the measurement, diagnosis, treatment, and management of overweight and obesity comprising the administration of the drug product to the patient (100) in need thereof to prevent or reduce the risk of metabolic syndrome and related disorders by using electronic devices according to any one of claims 16 to 21, wherein the pharmaceutical composition with the drug (130) comprises drug products for the prevention, reduction, or control of hyperglycemia (type I diabetes or type II diabetes), selected from the group consisting of: chemical uncouplers of oxidative phosphorylation or derivatives thereof, agents acting on the ATP-dependent potassium channel of pancreatic [beta] cells, biguanides, imidazolines, [alpha]-glucosidase inhibitors, meglitinides, oxadiazolidinediones, thiazolidinediones, insulin sensitizers, sulfonylureas, potassium channel blockers, glucagon antagonists, DPP-IV (dipeptidyl peptidase-IV) inhibitors, PTPase (protein tyrosine phosphatase) inhibitors, glucokinase activators, inhibitors of liver enzymes involved in the stimulation of gluconeogenesis and/or glycogenolysis, glucose uptake modulators, GSK-3 (glycogen synthase kinase-3) inhibitors, compounds that modify lipid metabolism, compounds that reduce food intake and PPAR agonists; tyrphostin AG17, tyrphostin A9, insulin and insulin analogs, tolbutamide, chlorpropamide, tolazamide, glibenclamide, glipizide, glimepiride, glicazide o gliburide, metformin, repaglinide or senaglinide/nateglinide, troglitazone, pioglitazone, rosiglitazone, isaglitazone, darglitazone, englitazone, GI 262570, YM-440, MCC-555, JTT-501, AR-H039242, KRP-297, GW-409544, CRE-16336, AR-H, voglibose, emiglitate, miglitol o acarbose, tolbutamide, glibenclamide, glipizide, glicazide, BTS-67582 o repaglinide, nateglinide, colestiramine, colestipol, clofibrate, gemfibrozil, lovastatine, pravastatine, simvastatine, pharmaceutically acceptable salts and its derivatives, and mixtures thereof.
